(19) United States
(12) Patent Application Publication
(10) Pub. No.: US 2021/0388060 A1
(43) Pub. Date: Dec. 16, 2021

(54) NOVEL METHOD FOR PRODUCING ANTIBODIES

(71) Applicant: TSINGHUA UNIVERSITY, Beijing (CN)

(72) Inventors: Bai LU, Beijing (CN); Hongyang YAO, Beijing (CN)

(21) Appl. No.: 17/289,244

(22) PCT Filed: Nov. 8, 2019
(86) PCT No.: PCT/CN2019/116592

§ 371 Date: Apr. 28, 2021

(30) Foreign Application Priority Data
Nov. 8, 2018 (CN) PCT/CN2018/114544

(51) Int. Cl. C07K 16/00; C12N 5/0783; C12N 5/0781; C12N 5/0784; C12N 5/077; C12N 5/0789; C12N 5/074; C12N 5/0775; A61K 35/17

(52) U.S. Cl. CPC C07K 16/00; C07K 2317/14; C12N 5/0635; C12N 5/0639; C12N 5/0653; C12N 5/0647; C12N 5/0696; C12N 5/0665; A61K 35/17; C12N 2501/2302; C12N 2501/2321; C12N 2501/2301; C12N 2501/21; C12N 2501/056; C12N 2501/52; C12N 5/0636

(57) ABSTRACT

Methods for producing an antibody or an antigen-binding fragment thereof specifically binding to an antigen of interest, methods for inducing proliferation of PBMCs, B cell activation and differentiation, B cell maturation, and/or promoting class switch in an antibody-producing PBMC to produce IgG, compositions for the in vitro immunization and methods for identifying an antibody-enhancing factor for in vitro immunization.

### FIELD OF THE INVENTION

The present disclosure generally relates to novel methods for producing antibodies, in particular in vitro method for producing fully human antibodies.

### BACKGROUND

Methods for producing antibodies are widely used in laboratory and clinics. Those include hybridoma technology, transgenic animal model and in vitro immunization. The traditional hybridoma technology is a mainstream mature technology, which includes steps of immunizing the animals, isolating lymphocyte, fusion of lymphocyte with immortalized cells such as myeloma, performing antibody humanization and affinity maturation. The antibodies can be produced in high throughput, but it has to face disadvantages including high cost, long production cycle, low affinity, unpredicted pair of heavy chain and light chain of the variable region. The transgenic animal model is a relatively new technology, where the animals are genetically modified to express human variable regions through unclear mechanisms. The in vitro immunization technology has been studied in recent years which does not require immunization of animals, and thus the process thereof are low in cost but faster and easier to operate, and the antibodies can be fully human without steps of humanization. However, few antibodies have been reported to be successfully generated using such methods. Therefore, there is a continuing need to develop new and effective methods for in vitro immunization to generate fully human antibodies.

### BRIEF SUMMARY OF THE INVENTION

The present disclosure provides a method for producing an antibody or an antigen-binding fragment thereof specifically binding to an antigen of interest, the method comprising:

mixing the antigen, an antibody-generating cell composition (AGC), and an antibody-enhancing composition in a medium to form a mixture,

cultivating the mixture,

obtaining the antibody from the mixture,

wherein the AGC comprises at least one B cell and at least one additional type of cell derived from peripheral blood mononuclear cells (PBMCs), and the antibody-enhancing composition comprises one or more adipose tissue-derived secretory proteins (ADSPs).

In certain embodiments, the antibody-enhancing composition further comprises IL2 and/or IL21.

In certain embodiments, the adipose tissue-derived secretory protein comprises one or more cytokines and/or one or more cell-adhesion molecules.

In certain embodiments, the cytokines comprise one or more interleukins and/or one or more chemokines. In certain embodiments, the interleukins are selected from a group consisting of IL1β, IL1f9, IL10, IL27, IL33, IL18BP, IL4, IL3, ILS, IL6, IL7, IL13, IL14, and IL15. In certain embodiments, the interleukins are selected from a group consisting of IL1β, IL1f9, IL10, IL27, IL33, IL18BP.

In certain embodiments, the chemokines comprise one or more CC-chemokines selected from a group consisting of CCL4, CCL8, CCL6, CCL9 and CCL11. In certain embodiments, the chemokines comprise one or more CXC-chemokines selected from a group consisting of CXCL2, CXCL5, CXCL16, CXCL9 and CXCL13.

In certain embodiments, the cytokines are selected from a group consisting of IL-1β, CCL8 and CXCL5. In certain embodiments, the cytokines comprise IL-1β and CCL8. In certain embodiments, the cytokines comprise CCL8 and CXCL5. In certain embodiments, the cytokines comprise IL-1β, CCL8 and CXCL5.

In certain embodiments, the cell-adhesion molecules are selected from a group consisting of ICAM1, CSF3r, Itgam, Siglecf, Adam8, Chl1, Sirpa, Nrcam, Emilin2, Emilin1, Tubb6, and/or Parvb.

In certain embodiments, the ADSP is derived from adipose tissue.

In certain embodiments, the AGC comprises at least one B cell and at least one T follicular helper cell. In certain embodiments, the AGC comprises at least one B cell and at least one dendritic cell. In certain embodiments, the AGC comprises at least one B cell, at least one T follicular helper cell and at least one dendritic cell. In certain embodiments, the AGC further comprises at least one adipocyte.

In certain embodiments, the AGC comprises PBMCs. In certain embodiments, the PBMCs are isolated from a blood sample, derived from human hematopoietic stem cells (HSCs), derived from induced pluripotent stem cells (iPSCs) or derived from umbilical cord blood.

In certain embodiments, the antibody-enhancing composition further comprises a co-stimulator, a toll-like receptor (TLR) agonist, a CpG oligodeoxynucleotide (CpG ODN), an anti-apoptotic protein, a TNF, an interferon (INF), a lipid, avasimid, EFNB1, EPHB4, Plexin B2, Semaphorin 4C, BLIMP-1, IRF4 or any combination thereof.

In certain embodiments, the co-stimulator comprises CD40, CD40L, ICOSL, ICOS, APRIL, B cell activating factor of the TNF family (BAFF), OX40, and/or OX40L.

In certain embodiments, the CpG ODN comprises CpG2006, and/or D/K CpG.

In certain embodiments, the anti-apoptotic protein comprises Bcl-2, Bcl-6, Bcl-XL, Bcl-w, Mc1-1, and/or an analog thereof.

In certain embodiments, the TLR agonist comprises a TLR1 agonist, a TLR2 agonist, a TLR3 agonist, a TLR4 agonist, a TLR5 agonist, a TLR6 agonist, a TLR7 agonist, a TLR8 agonist, a TLR7/8 agonist and/or a TLR9 agonist.

In certain embodiments, the method further comprises isolating the antibody from the mixture. In certain embodiments, the method further comprises obtaining a nucleic acid sequence encoding a variable region of the antibody. In certain embodiments, the method further comprises introducing the nucleic acid sequence into a host cell under a condition suitable for expressing the antibody or the antigen-binding fragment thereof. In certain embodiments, the method further comprises evaluating if the antibody specifically binds to the antigen of interest.

In certain embodiments, the ADSP is present at a concentration of at least 1 ng/ml, 10 ng/ml, or 50 ng/ml. In certain embodiments, IL2 is present at a concentration of at least 10 ng/ml. In certain embodiments, IL21 is present at a concentration of at least 50 ng/ml.

In certain embodiments, the ADSP is present for at least 1 day. In certain embodiments, the IL2 is present for at least 1 day. In certain embodiments, the IL21 is present for at least 1 day.

The present disclosure also provides herein a method for inducing proliferation of antibody-generating cell composition (AGC), B cell activation and differentiation, B cell maturation, and/or promoting class switch in an AGC to produce IgG, wherein the method comprising cultivating the AGC in a medium comprising IL2, an adipose tissue-derived secretory protein, and/or IL21. In certain embodiments, the AGC comprises PBMCs. In certain embodiments, an antigen of interest is present in the medium.

In certain embodiments, the antibody produced is a fully human monoclonal antibody.

Also provided herein is a method for producing an antibody or antigen-binding fragment thereof specifically binding to an antigen of interest, the method comprising:

mixing the antigen, an antibody-generating cell composition (AGC), and an antibody-enhancing composition in a medium to form a mixture,

cultivating the mixture,

obtaining the antibody from the mixture,

wherein the AGC comprises at least one B cell and at least one additional type of cell derived from peripheral blood mononuclear cells (PBMCs), and the antibody-enhancing composition comprises IL2, IL21, and one or more adipose tissue-derived secretory proteins (ADSPs).

In certain embodiments, the method further comprises obtaining a nucleic acid molecule encoding a variable region of the antibody from the mixture; and optionally introducing the nucleic acid molecule into a host cell under a condition suitable for expressing the antibody or the antigen-binding fragment thereof. In certain embodiments, the method further comprises isolating the antibody or the antigen-binding fragment thereof secreted by the host cell.

The present disclosure also provides herein a composition comprising isolated antibody-generating cell composition (AGC) comprising at least one B cell and at least one additional type of cell derived from peripheral blood mononuclear cells (PBMCs), an antibody-enhancing composition, and a medium. In certain embodiments, the composition further comprises an antigen of interest. In certain embodiments, the antibody-enhancing composition further comprises IL2 and/or IL21. In certain embodiments, the AGC comprises at least one B cell and at least one T follicular helper cell. In certain embodiments, the AGC comprises at least one B cells and at least one dendritic cell. In certain embodiments, the AGC comprises at least one B cell, at least one T follicular helper cell and at least one dendritic cell. In certain embodiments, the AGC comprises PBMCs.

In certain embodiments, the AGC further comprises at least one adipocyte.

In certain embodiments, the antibody-enhancing composition comprises one or more antibody-enhancing factor selected from the group consisting of ADSP, CD40L, ICOSL, ICOS, TLR agonist and any combination thereof

The present disclosure provides herein a method for identifying an antibody-enhancing factor for in vitro immunization, comprising:

a) isolating total RNA from a cell derived from a lymph node of an animal immunized with an antigen of interest;

b) comparing the RNA levels of the total RNA isolated from the step a) with that of a control animal without immunization to determine a gene which encodes a protein and whose expression level is upregulated;

c) cultivating PBMCs in a medium comprising the antigen of interest, IL2, IL21 and the protein;

d) identifying the protein as an antibody-enhancing factor for in vitro immunization if the protein enhances antibody production.

In certain embodiments, the cell is an adipocyte, a T follicular helper cell, a B cell or a dendritic cell.

In certain embodiments, the protein is expressed by the adipocyte, the T follicular helper cell, the B cell or the dendritic cell.

In one aspect, the present disclosure provides a method for producing a chimeric antigen receptor (CAR), comprising a step of expressing a first nucleic acid operably linked to a second nucleic acid, wherein the first nucleic acid encodes an antigen binding domain derived from the antibody or antigen-binding fragment thereof produced according to the method provided herein, and wherein the second nucleic acid encodes a T-cell signaling domain.

In one aspect, the present disclosure provides a method of treating a cancer in a subject comprising: expressing in a T cell a first nucleic acid operably linked to a second nucleic acid, wherein the first nucleic acid encodes an antigen binding domain derived from the antibody or antigen-binding fragment thereof produced according to the method provided herein, and wherein the second nucleic acid encodes a T-cell signaling domain; and administering the T cell to the subject. In certain embodiments, the T cell is obtained from the subject.

### BRIEF DESCFRIPTION OF FIGURES

FIG. 1 illustrates RNA-seq analysis of up-regulated genes in adipose cells infiltrated into lymph nodes after immunization.

FIG. 2 shows effect of IL-1β in stimulating the antibody production.

FIG. 3 shows effect of CCL8 in stimulating the antibody production.

FIG. 4 shows effect of CXCL5 in stimulating the antibody production.

FIG. 5 shows effects of CXCL13, CCL4, 1L27, CXCL1 6 and CXCL2 in stimulating the antibody production.

FIG. 6 shows treatment with IL-1β increases the specificity of antibody production without compromising efficiency. FIG. 6A and FIG. 6B respectively shows the production level of anti-OVA IgG and IgM antibodies in the presence or absence of OVA. FIG. 6C shows the production level of non-specific anti-BSA IgG antibody in the presence or absence of OVA.

FIG. 7 shows the expression level of the ADSPs IL-1β, CCL8, CXCL5, and IL36 in the mice in the presence or absence of OVA immunization.

### DETAILED DESCRIPTION OF THE INVENTION

The following description of the disclosure is merely intended to illustrate various embodiments of the disclosure. As such, the specific modifications discussed are not to be construed as limitations on the scope of the disclosure. It will be apparent to one skilled in the art that various equivalents, changes, and modifications may be made without departing from the scope of the disclosure, and it is understood that such equivalent embodiments are to be included herein. All references cited herein, including publications, patents and patent applications are incorporated herein by reference in their entirety.

### Definitions

The term “antibody” as used herein includes any immunoglobulin, monoclonal antibody, polyclonal antibody, multivalent antibody, multispecific antibody, or bispecific (bivalent) antibody or a functional portion thereof that binds to a specific antigen. A native intact antibody comprises two heavy chains (H) and two light (L) chains inter-connected by disulfide bonds. Each heavy chain consists of a variable region (VH) and a first, second, and third constant region (CH1, CH2 and CH3, respectively), while each light chain consists of a variable region (VL) and a constant region (CL). Mammalian heavy chains are classified as α, δ, ε, γ, and μ, and mammalian light chains are classified as λ or κ. The variable regions of the light and heavy chains are responsible for antigen binding. The variables region in both chains are generally subdivided into three regions of hypervariability called the complementarity determining regions (CDRs) (light (L) chain CDRs including LCDR1, LCDR2, and LCDR3, heavy (H) chain CDRs including HCDR1, HCDR2, HCDR3). CDR boundaries for the antibodies and antigen-binding fragments disclosed herein may be defined or identified by the conventions of Kabat, Chothia, or Al-Lazikani (Al-Lazikani, B., Chothia, C., Lesk, A. M., J. Mol. Biol., 273(4), 927 (1997); Chothia, C. et al., J Mol Biol. December 5; 186(3):651-63 (1985); Chothia, C. and Lesk, A. M., J. Mol. Biol., 196,901 (1987); Chothia, C. et al., Nature. December 21-28; 342(6252):877-83 (1989) ; Kabat E. A. et al., National Institutes of Health, Bethesda, Md. (1991)). The three CDRs are interposed between flanking stretches known as framework regions (FRs), which are more highly conserved than the CDRs and form a scaffold to support the hypervariable loops. Therefore, each V_{H} and V_{L} comprises of three CDRs and four FRs in the following order (amino acid residues N terminus to C terminus): FR1, CDR1, FR2, CDR2, FR3, CDR3, FR4. The constant regions of the heavy and light chains are not involved in antigen binding, but exhibit various effector functions. Antibodies are assigned to the five major classes based on the amino acid sequence of the constant region of their heavy chain: IgA, IgD, IgE, IgG, and IgM, which are characterized by the presence of α, δ, ε, γ, and μ heavy chains, respectively. Subclasses of several of the major antibody classes are such as IgG1 (γ1 heavy chain), IgG2 (γ2 heavy chain), IgG3 (γ3 heavy chain), IgG4 (γ4 heavy chain), IgA1 (α1 heavy chain), or IgA2 (α2 heavy chain).

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods.

The term “fully human antibody” as used herein, with reference to antibody or antigen-binding fragment, means that the antibody or the antigen-binding fragment has or consists of amino acid sequence(s) corresponding to that of an antibody produced by a human or a human immune cell, or derived from a non-human source such as a transgenic non-human animal that utilizes human antibody repertoires or other human antibody-encoding sequences. In certain embodiments, a fully human antibody does not comprise amino acid residues (in particular antigen-binding residues) derived from a non-human antibody.

An “affinity matured” antibody refers to an antibody with one or more alterations or substitutions with amino acid residues in one or more hypervariable regions (HVRs), such as the complementarity determining regions (CDRs), compared to a parent antibody without such alterations or substitutions, which confer an improvement in the affinity of the antibody for antigen.

As used herein, the term “antigen-binding fragment” refers to an antibody fragment formed from a fragment of an antibody comprising one or more CDRs, or any other antibody portion that binds to an antigen but does not comprise an intact native antibody structure. In certain embodiments, the antibody provided herein is an antigen-binding fragment. Examples of antigen-binding fragment include, without limitation, a diabody, a Fab, a Fab′, a F(ab′)₂, an Fv fragment, a disulfide stabilized Fv fragment (dsFv), a (dsFv)₂, a bispecific dsFv (dsFv-dsFv'), a disulfide stabilized diabody (ds diabody), a single-chain antibody molecule (scFv), an scFv dimer (bivalent diabody), a multispecific antibody, a camelized single domain antibody, a nanobody, a domain antibody, an isolated CDR and a bivalent domain antibody. An antigen-binding fragment is capable of binding to the same antigen to which the parent antibody binds. In certain embodiments, an antigen-binding fragment may comprise one or more CDRs from a particular human antibody.

An “antigen” or “Ag” as used herein refers to a compound, composition, peptide, polypeptide, protein, RNA, DNA, a bacteria, a virus, or any immunogenic substance that can stimulate the production of antibodies or a T cell response in cell culture or in an animal, including compositions that are added to a cell culture (such as a hybridoma), or injected or absorbed into an animal. An antigen reacts with the products of specific humoral or cellular immunity (such as an antibody), including those induced by heterologous antigens.

“Fab” with regard to an antibody refers to a monovalent antigen-binding fragment of the antibody consisting of a single light chain (both variable and constant regions) bound to the variable region and first constant region of a single heavy chain by a disulfide bond. Fab can be obtained by papain digestion of an antibody at the residues proximal to the N-terminus of the disulfide bond between the heavy chains of the hinge region.

“Fab”' refers to a Fab fragment that includes a portion of the hinge region, which can be obtained by pepsin digestion of an antibody at the residues proximal to the C-terminus of the disulfide bond between the heavy chains of the hinge region and thus is different from Fab in a small number of residues (including one or more cysteines) in the hinge region.

“F(ab')2” refers to a dimer of Fab' that comprises two light chains and part of two heavy chains.

“Fc” with regard to an antibody refers to that portion of the antibody consisting of the second and third constant regions of a first heavy chain bound to the second and third constant regions of a second heavy chain via disulfide bond. IgG and IgM Fc regions contain three heavy chain constant regions (second, third and fourth heavy chain constant regions in each chain). It can be obtained by papain digestion of an antibody. The Fc portion of the antibody is responsible for various effector functions such as ADCC, and CDC, but does not function in antigen binding.

“Fv” with regard to an antibody refers to the smallest fragment of the antibody to bear the complete antigen binding site. A Fv fragment consists of the variable region of a single light chain bound to the variable region of a single heavy chain. A “dsFv” refers to a disulfide-stabilized Fv fragment that the linkage between the variable region of a single light chain and the variable region of a single heavy chain is a disulfide bond.

“Single-chain Fv antibody” or “scFv” refers to an engineered antibody consisting of a light chain variable region and a heavy chain variable region connected to one another directly or via a peptide linker sequence (Huston J S et al. Proc Natl Acad Sci USA, 85:5879(1988)). A “scFv dimer” refers to a single chain comprising two heavy chain variable regions and two light chain variable regions with a linker. In certain embodiments, an “scFv dimer” is a bivalent diabody or bivalent ScFv (BsFv) comprising V_{H}-V_{L} (linked by a peptide linker) dimerized with another V_{H}-V_{L} moiety such that V_{H}'s of one moiety coordinate with the V_{L}'s of the other moiety and form two binding sites which can target the same antigens (or eptipoes) or different antigens (or eptipoes). In other embodiments, a “scFv dimer” is a bispecific diabody comprising V_{H1}-V_{L2} (linked by a peptide linker) associated with V_{L1}-V_{H2} (also linked by a peptide linker) such that V_{H1} and V_{L1} coordinate and V_{H2} and V_{L2} coordinate and each coordinated pair has a different antigen specificity.

“Single-chain Fv-Fc antibody” or “scFv-Fc” refers to an engineered antibody consisting of a scFv connected to the Fc region of an antibody.

“Camelized single domain antibody,” “heavy chain antibody,” “nanobody” or “HCAb” refers to an antibody that contains two V_{H} domains and no light chains (Riechmann L. and Muyldermans S., J Immunol Methods. December 10; 231(1-2):25-38 (1999); Muyldermans S., J Biotechnol. June; 74(4):277-302 (2001); WO94/04678; WO94/25591; U.S. Pat. No. 6,005,079). Heavy chain antibodies were originally obtained from Camelidae (camels, dromedaries, and llamas). Although devoid of light chains, camelized antibodies have an authentic antigen-binding repertoire (Hamers-Casterman C. et al., Nature. June 3; 363(6428):446-8 (1993); Nguyen V K. et al. “Heavy-chain antibodies in Camelidae; a case of evolutionary innovation,” Immunogenetics. April; 54(1):39-47 (2002); Nguyen V K. et al. Immunology. May; 109(1):93-101 (2003)). The variable domain of a heavy chain antibody (VHH domain) represents the smallest known antigen-binding unit generated by adaptive immune responses (Koch-Nolte F. et al., FASEB J. November; 21(13):3490-8. Epub 2007 Jun. 15 (2007)). “Diabodies” include small antibody fragments with two antigen-binding sites, wherein the fragments comprise a V_{H} domain connected to a V_{L} domain in a single polypeptide chain (V_{H}-V_{L} or V_{L}-V_{H}) (see, e.g., Holliger P. et al., Proc Natl Acad Sci USA. July 15; 90(14):6444-8 (1993); EP404097; WO93/11161). The two domains on the same chain cannot be paired, because the linker is too short, thus, the domains are forced to pair with the complementary domains of another chain, thereby creating two antigen-binding sites. The antigen-binding sites may target the same of different antigens (or epitopes).

A “domain antibody” refers to an antibody fragment containing only the variable region of a heavy chain or the variable region of a light chain. In certain embodiments, two or more V_{H} domains are covalently joined with a peptide linker to form a bivalent or multivalent domain antibody. The two V_{H} domains of a bivalent domain antibody may target the same or different antigens.

In certain embodiments, a “(dsFv)₂” comprises three peptide chains: two V_{H} moieties linked by a peptide linker and bound by disulfide bridges to two V_{L} moieties.“Substantially”, “substantially the same” as used herein refer to a high degree of similarity between two numeric values, and those skilled in the art would not recognize or consider a significant difference between the two values or of little difference with regard to statistics and/or biological activity as indicated by the values. In contrast, “substantially lower” means that a numeric value is less than about 50%, less than about 40%, less than about 30%, less than about 20%, less than about 10% as a function of the reference value.

The term “specific binding” or “specifically binds” as used herein refers to a non-random binding reaction between two molecules, such as for example between an antibody and an antigen. In certain embodiments, the antibodies or antigen-binding fragments provided herein specifically bind human and/or non-human antigen with a binding affinity (K_{D}) of about 0.01 nM to about 100 nM, about 0.1 nM to about 100 nM, 0.01 nM to about 10 nM, about 0.1 nM to about 10 nM, 0.01 nM to about 5 nM, about 0.1 nM to about 5 nM, 0.01 nM to about 1 nM, about 0.1 nM to about 1 nM or about 0.01 nM to about 0.1 nM). K_{D} as used herein refers to the ratio of the dissociation rate to the association rate (k_{off}/kₒₙ), may be determined using surface plasmon resonance methods for example using instrument such as Biacore.

“Treating”, “treatment” or “therapy” of a condition as used herein can be used interchangeably, and includes therapeutic treatment, prophylactic or preventative measures, such as preventing or alleviating a condition, slowing the onset or rate of development of a condition, reducing the risk of developing a condition, preventing or delaying the development of symptoms associated with a condition, reducing or ending symptoms associated with a condition, generating a complete or partial regression of a condition, curing a condition, or some combination thereof.

The term “vector” as used herein refers to a vehicle into which a polynucleotide encoding a protein may be operably inserted and transported so as to express that protein in a host cell. A vector may be used to transform, transduce, or transfect a host cell so as to bring about the expression of the genetic element it carries within the host cell. Exemplary types of vectors includes, but not limited to, plasmids (e.g. phagemids, cosmids, yeast artificial chromosome (YAC), bacterial artificial chromosome (BAC) or P1-derived artificial chromosome (PAC)), viral vector (bacteriophages such as lambda phage or M13 phage, or animal viruses), bacterial vector, or non-episomal mammalian vectors. Categories of animal viruses used as vectors include retrovirus (including lentivirus), adenovirus, adeno-associated virus, herpesvirus (e.g., herpes simplex virus), poxvirus, baculovirus, papillomavirus, and papovavirus (e.g., SV40). A vector may contain a variety of elements for controlling expression, including promoter sequences, transcription initiation sequences, enhancer sequences, selectable elements, and reporter genes. In addition, the vector (e.g. a bacterial vector or episomal mammalian vector) may contain an origin of replication. A vector may also include materials to aid in its entry into the cell, including but not limited to a viral particle, a liposome, or a protein coating.

A “nucleic acid” or a “nucleic acid sequence” or “polynucleotide”, can be used interchangeably herein, refers to deoxyribonucleic acids (DNA) or ribonucleic acids (RNA) and polymers thereof in either single- or double-stranded form. Unless specifically limited, the term encompasses polynucleotides containing known analogues of natural nucleotides that have similar binding properties as the reference nucleic acid and are metabolized in a manner similar to naturally occurring nucleotides. Unless otherwise indicated, a particular polynucleotide sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The term “host cell” as used herein refers to a cell into which an exogenous polynucleotide and/or a vector has been introduced to express one or more exogenous proteins. It intends to refer to both the particular subject cell and the progeny thereof. A host cell can be a prokaryote, a eukaryote, a plant cell, an animal cell or a hybridoma. It can be a cell that does not express a protein at a desired level but comprises the nucleic acid, unless a regulatory agent is introduced into the cell or a regulatory sequence is introduced into the host cell so that it is operably linked with the nucleic acid.

The term “animal” as used herein refers to a mammal, for example, a human, a camelidae, a mouse, a rat, a rabbit, a goat, a sheep, a guinea pig, or a hamster. In certain embodiment, the animal is a human.

An “isolated” substance has been altered by the hand of man from the natural state. If an “isolated” composition or substance occurs in nature, it has been changed or removed from its original environment, or both. For example, an “isolated” polynucleotide or polypeptide is a polynucleotide or a polypeptide that is free of other polynucleotides or polypeptides, respectively, and is not associated with naturally components that accompany the polynucleotide or a polypeptide in the native state. In certain embodiments, an “isolated” protein is purified by at least one step to a purity of at least 90%, 91%, 92%, 93%, 94%, 95%, 96%, 97%, 98%, 99% as determined by electrophoretic methods (such as SDS-PAGE using Coomassie blue or silver stain, isoelectric focusing, capillary electrophoresis), chromatographic methods (such as ion exchange chromatography or reverse phase HPLC) or Lowry method.

Activation-induced cytidine deaminase, also known as AICDA and AID, is a 24 kDa enzyme which in humans is encoded by the AICDA gene. AID is a member of the cytidine deaminase family that is involved in somatic hypermutation and class-switch recombination of immunoglobulin genes in B cells and is thought to be the master regulator of secondary antibody diversification. AID generates DNA mutations and turns cytosine to uracil (recognized as thymine during DNA replication), converting C:G to T:A or A:T base pair during germinal center development of B lymphocytes. During somatic hypermutation, the antibody is mutated to generate a library of antibody variants with various affinities.

PR domain zinc finger protein 1 is also known as BLIMP-1, which is a transcriptional repressor protein encoded by the PRDM1 gene in humans. BLIMP-1 binds specifically to the PRDI (positive regulatory domain I element) of the beta-interferon (beta-IFN) gene promoter and represses gene expression of beta-IFN. Increased BLIMP-1 protein in B lymphocytes, T lymphocytes, NK cell and other immune cells leads to an immune response through proliferation and differentiation of antibody secreting plasma cells.

The present disclosure provides a method of in vitro immunization, induction of a humoral response in vitro, i.e. the in vitro production of antigen-specific human antibodies which results from the recognition of said antigen by the immunoglobulins expressed at the surface of naive human B lymphocytes cultured, in vitro, with the antigen.

In animal immunization, germinal centers (GCs) are important sites within lymph nodes and the spleen, wherein mature B cells proliferate, differentiate, and mutate their antibody genes through somatic hypermutation to achieve higher affinity, and switch the class of antibody from IgM to IgG during an immune response. GCs are important in B cell humoral immune response as the center of generation of affinity matured B cells and durable memory B cells. In the GCs, the B cells undergo rapid and mutative cellular division in the dark zone (where they are called centroblasts) and migrate to the light zone (where they are called centrocytes), where they are subject to selection by follicular helper T cells in the presence of follicular dendritic cells. Those selected B cells return to the dark zone to further undergo division and mutation. In the meantime, small amount of memory B cells and plasma cells depart the GCs.

Different from the GCs in the animal, the in vitro immunization methods are capable of generating antibodies in in vitro GC like B cells, which are contained in peripheral blood mononuclear cells (PBMCs).

Antibody-Generating Cell Composition (AGC)

The term “antibody-generating cell composition” or “AGC” refers to a group of cells that generates antibodies against an antigen of interest under a suitable condition for the antibody production. In certain embodiments, the AGC comprises at least one B cell and at least one additional type of cell that is derived from peripheral blood mononuclear cells (PBMCs). In certain embodiments, the AGC comprises PBMCs.

The peripheral blood mononuclear cells (PBMCs) are any peripheral blood cell having a round nucleus, comprising lymphocytes and monocytes. The PBMC can be extracted from whole blood by conventional techniques in the art, such as density gradient centrifugation using ficoll, a hydrophilic polysaccharide that separates layers of blood, and gradient centrifugation, which will separate the blood into a top layer of plasma, followed by a layer of PBMCs and a bottom fraction of polymorphonuclear cells (such as neutrophils and eosinophils) and erythrocytes. Proliferation of PBMCs can be detected or confirmed in vitro by methods known in the art, for example, by MTT assay (a colorimertic method), AO/PI (Acridine Orange and Propidium Iodide) staining, or cell counting. In certain embodiments, the PBMCs are isolated from the whole blood sample. In certain embodiments, the PBMCs are derived from human hematopoietic stem cells (HSCs), derived from induced pluripotent stem cells (iPSCs) or derived from umbilical cord blood.

In certain embodiments, the AGC is a mixture of isolated cell types comprising lymphocytes (T cells, B cells, NK cells), monocytes, macrophages and dendritic cells.

In certain embodiments, the AGC comprises PBMCs. In certain embodiments, the AGC comprises at least one B cell. In certain embodiments, the AGC comprises at least one of B cell, at least one T cell (e.g. T follicular helper cell), at least one dendritic cell, at least one NK cell, at least one monocyte, and at least one adipocyte.

For example, in certain embodiments, the AGC comprises at least one B cell and at least one T cell (e.g. T follicular helper cell). In certain embodiments, the AGC comprises at least one B cell and at least one dendritic cell. In certain embodiments, the AGC comprises at least one B cell, at least one T cell (e.g. T follicular helper cell), and at least one dendritic cell. In certain embodiments, the AGC comprises at least one B cell and at least one NK cell. In certain embodiments, the AGC comprises at least one B cell and at least one monocyte. In certain embodiments, the AGC comprises at least one B cell, T cell (e.g. T follicular helper cell), and at least one NK cell. In certain embodiments, the AGC comprises at least one B cell, at least one T cell (e.g. T follicular helper cell), at least one dendritic cell and at least one NK cell.

In certain embodiments, the AGC comprises at least one adipocyte and at least one B cell. In certain embodiments, the AGC comprises at least one adipocyte, at least one B cell and at least one T cell (e.g. T follicular helper cell). In certain embodiments, the AGC comprises at least one adipocyte, at least one B cell, and at least one dendritic cell. In certain embodiments, the AGC comprises at least one adipocyte, at least one B cell, at least one T cell (e.g. T follicular helper cell), and at least one dendritic cell.

In certain embodiments, at least one of the B cells, T follicular helper cells, dendritic cells, and adipocytes are human cells. In certain embodiments, the B cells are human B cells. In certain embodiments, the PBMCs are derived from human PBMCs.

In certain embodiments, the PBMCs are isolated from a human donor. In certain embodiments, the PBMCs are derived from stem cells.

The term “B cell” as used herein refers to B lymphocytes, a type of white blood cell of the lymphocyte subtype. They function in the humoral immunity component of the adaptive immune system by secreting antibodies. B cells also present antigen and secrete cytokines. In mammals, B cells mature in the bone marrow. After B cells mature in the bone marrow, they migrate through the blood to secondary lymphoid organs (SLOs), such as the spleen and lymph nodes, where B cells receive a constant supply of antigen through circulating lymph. Unlike the other two classes of lymphocytes, i.e. T cells and natural killer cells, B cells express B cell receptors (BCRs) on their cell membrane, which allow the B cell to bind a specific antigen, against which it will initiate an antibody response. Of the three B cell subsets, FO B cells preferentially undergo T cell-dependent (TD) activation while marginal zone (MZ) B cells and B1 B cells preferentially undergo T cell-independent (TI) activation. B cells activated by TI antigens proliferate outside of lymphoid follicles but still in SLOs, possibly undergo immunoglobulin class switching, and differentiate into short-lived plasmablasts that produce early, weak antibodies mostly of class IgM, but also some populations of long-lived non-proliferating antibody-producing plasma cells. B cell activation is enhanced through the activity of CD21, a surface receptor in complex with surface proteins CD19 and CD81 (all three are collectively known as the B cell co-receptor complex, or BCR). When a BCR binds an antigen tagged with a fragment of the C3 complement protein, CD21 binds the C3 fragment, co-ligates with the bound BCR, and signals are transduced through CD19 and CD81 to lower the activation threshold of the cell.

In certain embodiments, the B cells are those naturally exist in the PBMCs from a healthy donor. In certain embodiments, the B cells are cultured B cells, differentiated B cells from stem cells, or isolated B cells isolated from animals. In certain embodiments, the B cells are genetically-engineered B cells to produce non-naturally occurred antibodies, such as bispecific antibodies.

The term “naive B lymphocytes” is intended to mean B lymphocytes (B cells) which have never encountered the antigen that they could bind via the paratope expressed by their surface immunoglobulin. These B cells are derived directly from the peripheral blood of a subject who has never been in contact with the antigen. These subjects will therefore exhibit a seronegative status with respect to said antigen, i.e. they will exhibit an undetectable titer of serum antibodies specific for said antigen.

The term “T cell” used herein refers to a lymphocyte which is derived from thymus and is mainly involved in cell immunity. Examples of the T cells include a CD4⁺ T cell (T helper cell, TH cell), a CD8⁺ T cell (cytotoxic T cell, CTL), a memory T cell, a regulatory T cell (Treg cell, such as activated Treg and unactivated Treg), an apoptotic T cell, a naive T cells, or other T cell populations.

In certain embodiments, the T cells are those naturally exist in the PBMCs from a healthy donor. In certain embodiments, the T cells are cultured T cells, differentiated T cells from stem cells, or isolated T cells isolated from animals. In certain embodiments, the T cells are genetic-engineered T cells.

“T helper cells” are a type of T cells involved in adaptive (that is, tailored to the specific pathogen) immune system via releasing T cell cytokines, thereby suppress or regulate immune responses. T helper cells are involved in B cell antibody class switching, activation and growth of cytotoxic T cells, and maximizing bactericidal activity of phagocytes such as macrophages. Mature T helper cells are CD4 positive and aid the antigen-presenting cells (APCs, such as dendritic cells) to express antigen on MEC class II, via combination of cytokines release and cell to cell interaction (e.g. CD40 (on APC) and CD40L (on T follicular helper cell)). T helper cells can develop into two major subtypes, Th1 and Th2 cells. Thl helper cells are involved in cellular immune system against intracellular bacteria and protozoa, and are triggered by IL-12 and release IFN-gamma and IL-2. Thl helper cells help enhance killing efficacy of macrophages, proliferation of CD8⁺ T cells, IgG-production of B cells, and ITN-gamma-secrecting CD4⁺ T cells. Th2 helper cells are involved in humoral immune system against extracellular parasites, and are triggered by IL-4 and IL-2 and release IL-4, IL-5, IL-9, IL-10, IL-13 and IL-25. Th2 helper cells help eosinophils, basophils, mast cells, stimulate B cells to proliferate and to produce antibodies, and IL-4/IL-5-secreting CD4⁺ T cells. T follicular helper cell are found in the periphery within B cell follicles of secondary lymphoid organs such as lymph nodes, spleens and Peyer's patches, and are identified by their constitutive expression of the B cell follicle homing receptor CXCR5. TFH cells trigger the formation and maintenance of germinal centers through the expression of CD40L and the secretion of IL-21 and IL-4 upon cellular interaction and cross-signaling with their cognate follicular (Fo B) B cells.

The term “antigen-presenting cell” or APC, is intended to mean a cell expressing one or more molecules of the class I and class II major histocompatibility complex (MHC) (class I and class II HLA molecules in humans) and capable of presenting antigens to CD4⁺ T and CD8⁺ T lymphocytes specific for this antigen. As antigen-presenting cells, mention may in particular be made of dendritic cells (DCs), peripheral blood mononuclear cells (PBMCs), monocytes, macrophages, B lymphocytes, lymphoblastoid lines, and genetically modified human or animal cell lines expressing class I and class II MHC molecules, in particular HLA I and HLA II molecules.

The term “cytotoxic T cells”, “T-killer cells” or “CTL” used herein is exchangeable and refers to a type of T cells that recognize a specific antigen produced by cancer cells, infected cells by viruses, or cells damaged in other ways. The antigens are brought to the surface of a cell by MHC class I, which is bound by the TCR on cytotoxic T cells in the aid of CD8. Thus, cytotoxic T cells are CD8 positive.

Memory T cells are a subset of T cells that have previously experienced (encountered and responded to) the antigens of cancer cells, bacteria or viruses. The memory T cells can be CD4⁺ and/or CD8⁺ T cells, or memory cytotoxic T cells. Upon re-exposure to an antigen, long-lived memory T cells can mediate a more rapid and more efficient secondary response. This memory function can be provided by CD4⁺ and/or CD8⁺ memory T cells. Long-lived memory T cells are different from effector cells that only have a short life time and usually die after an immune response by activation-inducing cell death (AICD). Between the two cell types, however, there are transitional forms, such as the effector memory cells. Like effector cells, they are able to patrol throughout the body, and exert an effector function upon antigen contact, and they can proliferate and are also more long-lived than effector cells.

“Regulatory T cells” or “Tregs” used herein refers to a subpopulation of T cells that modulate the immune system, maintain tolerance to self-antigens and prevent autoimmune response. Tregs are immnosuppressive and is involved in inhibition of self-reactive immune responses. Tregs are CD4, CLTA4, GITR, neuropilin-1, and CD25 positive. Tregs perform their suppressive function on activated T cells through contact-dependent mechanisms and cytokine production (Fehervari, Z. & Sakaguchi, Curr Opin Immunol 16, 203-8 (2004)). Tregs also modulate immune responses by direct interaction with ligands on dendritic cells (DC), such as CTLA4 interaction with B7 molecules on DC that elicits the induction of indoleamine 2,3-dioxygenase (IDO) (Fallarino, F. et al., Nat Immunol 4, 1206-12 (2003)), and CD40L ligation (Serra, P. et al., Immunity 19, 877-89 (2003)).

“Natural Killer (NK) cells” as used herein refer to lymphocytes which typically have CD16 and/or and/or NCAM and/or CD56 molecules expressed as cell surface markers but which do not express CD3. The NK cells refer to cells present in vivo in a mammal or in vitro in the form of a purified population of cells. NK cells are a type of cytotoxic lymphocyte critical to the innate immune system. The role of NK cells is analogous to that of cytotoxic T cells.

“Dendritic cells (DCs)” are potent antigen-presenting cells (APCs) that process antigen material and present it on the cell surface to the T cells. Upon activation, DCs migrate to the lymph nodes where they interact with T cells and B cells to initiate and shape the adaptive immune response. Human dendritic cells selectively express CD83. DCs have a variety of surface receptors with which they can identify various pathogens. In addition, DCs are able to perceive various endogenous messengers such as cytokines and chemokines, and surface molecules on other cells of the immune system. The DCs process the various incoming signals via intracellular signaling pathways, whereby various differentiation programs are triggered. Dendritic cells are able to initiate primary T cell responses in vitro and in vivo. DCs can be produced ex vivo and loaded with various protein and peptide antigens as well as tumor cell extracts (Nestle, F. et al., Nat. Med., 4:328-332 (1998)). DCs may also be transduced by genetic means to express these tumor antigens as well. DCs have also been fused directly to tumor cells for the purposes of immunization (Kugler, A. et al., Nat. Med., 6:332-336 (2000)).

In certain embodiments, the DCs are those naturally exist in the PBMCs from a healthy donor. In certain embodiments, the DCs are cultured DCs, differentiated DCs from stem cells, or isolated DCs isolated from animals. In certain embodiments, the T cells are genetic-engineered DCs.

Adipocytes refers to lipocytes and fat cells, are the cells that primarily compose adipose tissue. The lymphatic peripheral adipocytes are found to tightly associate with and even infiltrate into lymph nodes after immunization to an animal. An adipose tissue-derived secretory protein (ADSP) refers to a protein within or secreted by the lymph node-associated adipose tissues that is capable of enhancing the antibody production by the AGC provided herein. An ADSP can be recognized after antigen immunization to an animal when its protein level or its RNA level is up-regulated to at least two-fold as compared with that of an animal without the antigen immunization or of the same animal prior to the antigen immunization. Although ADSP is secreted by an adipocyte, it can also be produced and secreted by other cell types derived from peripheral blood mononuclear cells (PBMCs), such as a B cell, a T cell (e.g. a T follicular helper cell), or a dendritic cell.

In certain embodiments, the adipocytes are cultured adipocytes, differentiated adipocytes from stem cells, or isolated adipocytes isolated from animals.

At least one type of the mononuclear cells, such as B cells, T cells (e.g. T follicular helper cell), dendritic cells, NK cells, monocytes, can be isolated from the whole blood of a subject, and/or reconstructed from hematopoietic stem cells (HSCs), bone marrow, new born umbilical cord blood (thus called cord blood mononuclear cells (CBMCs)), amniotic fluid, or pluripotent stem cells (hPSCs, comprising both embryonic stem cells (ESCs) and induced pluripotent stem cells (iPSCs)). In certain embodiments, at least one type of the mononuclear cells can be from an adult, adolescent or child.

The hematopoietic stem cells (HSCs) are located in the red bone marrow and generates various type of mature blood cells during the haematopoiesis, including myeloid cells (monocytes, macrophages, neutrophils, basophils, eosinophils, erythrocytes, dendritic cells, and megakaryocytes or platelets) and lymphoid cells (T cells, B cells, and natural killer cells). Bone marrow is the spongy or cancellous, semi-solid tissue in the bone that composed of hematopoietic cells (myeloid and lymphoid lineages), marrow adipose tissue, mesenchymal stem cells (MSCs) and supportive stromal cells. Human bone marrow typically produces around 500 billion blood cells per day that enter into circulation via permeable vasculature sinusoids within the medullary cavity. The lymphoid cells mature in other lymphoid organs, such as thymus.

Umbilical cord blood comprises numerous immunologically immature newborn umbilical cord blood mononuclear cells (UCBMCs) and is also reported a source of hematopoietic stem cells (see Gluckman E et al., Hematopoietic reconstitution in a patient with Fanconi's anemia by means of umbilical-cord blood from an HLA-identical sibling.N Engl J Med. 1989 Oct. 26; 321(17):1174-8.). The mononuclear cells and/or HSCs can be differentiated from human pluripotent stem cells (hPSCs, comprising both human embryonic stem cells (ESCs) and induced pluripotent stem cells (iPSCs)) in vitro, such as primitive hematoendothelial precursors, mature myeloid, erythroid, and lymphoid lineage cells (Melinda K. Hexum et al., In Vivo Evaluation of Putative Hematopoietic Stem Cells Derived from Human Pluripotent Stem Cells, Human Pluripotent Stem Cells, 2011. pp 433-447). Amniotic fluid also contains mononuclear cells and cells with hematopoietic activity (see Ditadi A et al., Human and murine amniotic fluid c-Kit+Lin-cells display hematopoietic activity, Blood. 2009 Apr. 23; 113(17):3953-60).

The antibody-producing cells provided herein used to produce the antibody or the antigen-binding fragment thereof may be cultured in a variety of media. Commercially available media such as Ham's F10 (Sigma), Minimal Essential Medium (MEM), (Sigma), RPMI-1640 (Sigma), and Dulbecco's Modified Eagle's Medium (DMEM), Sigma) are suitable for culturing the antibody-generating cell composition (AGC). In addition, any of the media described in Ham et al. , Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the antibody-generating cell composition (AGC). Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with antibody-generating cell composition (AGC) selected for expression, and will be apparent to the ordinarily skilled artisan.

Antibody-Enhancing Composition

The term “antibody-enhancing composition” as used herein, refers to a collection of factors that are capable of enhancing the amount of antibodies produced in an in vitro antibody production system. An antibody-enhancing composition is capable of inducing proliferation of PBMCs, B cell activation and differentiation, B cell maturation, T cell differentiation, maturation of antibody affinity, antibody diversity and/or promoting class switch in an antibody-producing PBMC to produce IgG in the in vitro immunization. In certain embodiments, the antibody-enhancing composition comprises one or more antibody-enhancing factors.

In certain embodiments, the antibody-enhancing composition or the antibody-enhancing factor is selected from the group consisting of an adipose tissue-derived secretory protein (ADSP), CD40- and CD40L-interacting compounds, ICOS- and ICOS-L-interacting compounds, TLR agonists, OX40, OX4OL, APRIL (a proliferation-inducing ligand), BAFF, CR2, chemokines (CXCL5, CXCL16, CXCL9, CXCL12 (SDF-1), CXCL13, CXCL16), Flt-3L, interleukins (IL1 (α/β), IL2, IL3, IL4, IL5, IL6, IL10, IL13, IL14, IL15, IL21, IL27, IL33, Th1f9, IL18BP), SAP (signaling lymphocyte activation molecule [SLAM] associated protein), Staphylococcus A strain Cowan 1 particles (SAC; heat-killed, formalin-fixed), TLR Ligands such as lipopolysaccharide (LPS), different CpG ODNs or Resiquimod (R-848), TSLP, Tumor necrosis factor (TNF) alpha, type I Interferons (e.g. IFN α/β), type II interferon (e.g. IFNγ), lipids, avasimid, EFNB1, EPHB4 (Lu et al., Science, 2017, eaai9264), Plexin B2, semaphoring 4C (Hu et al., Cell Reports, 2017, 19, 995-1007), BLIMP-1, IRF4, cell-adhesion molecules (ICAM1, CSF3r, Itgam, Siglecf, Adam8, Chl1, Sirpa, Nrcam, Emilin2, Emilinl, Tubb6, and Parvb), and any combination thereof.

In certain embodiments, the antibody-enhancing composition or the antibody-enhancing factor is selected from an adipose tissue-derived secretory protein (ADSP), a CD40L, an ICOSL, an ICOS, a TLR agonist and any combination thereof.

In certain embodiments, the adipose tissue-derived secretory protein enhances antibody production by the AGC, activation and differentiation of the B cell in the AGC, and/or maturation of the B cell in the AGC.

In certain embodiments, the ADSP level or its RNA level in the immunized animal is 3-, 4-, 5-, 6-, 7-, 8-, 9-, 10-, 15-, 20-, 25-, 30-, 35-, 40-, 45- or 50-fold higher than the control animal without immunization, or the same animal prior to the immunization.

The antibody-enhancing factor is intended to encompass any form, for example, 1) native unprocessed molecule, “full-length” molecule chain or naturally occurring variants of the molecule, including, for example, splice variants or allelic variants; 2) any form that results from processing in the cell; or 3) full length, a fragment (e.g., a truncated form, an antibody-enhancing domain); a modified form (e.g. a mutated form, a glycosylated/PEGylated, a His-tag/immunofluorescence fused form) generated through recombinant method; or 4) homologs in other species.

In certain embodiments, the ADSP comprises cytokines and cell-adhesion molecules that are capable of enhancing antibody production. In certain embodiments, the AD SP is capable of enhancing IgG percentage in the total antibody production.

Cytokines are small proteins (-5-20 kDa) that are important in cell signaling that includes autocrine signaling, paracrine signaling and endocrine signaling as immunomodulating agents. Cytokines can be produced by immune cells such as macrophages, B lymphocytes, T lymphocytes and mast cells, as well as endothelial cells, fibroblasts, and various stromal cells. Cytokines include chemokines, interferons, interleukins, and tumour necrosis factors (TNF).

Interferons (IFNs) belong to the large class of proteins known as cytokines, molecules used for communication between cells to trigger the protective defenses of the immune system that help eradicate pathogens. IFNs are a group of signaling proteins made and released by host cells in response to the presence of several pathogens, such as viruses, bacteria, parasites, and also tumor cells. IFNs also have various other functions: they activate immune cells, such as natural killer cells and macrophages; they increase host defenses by up-regulating antigen presentation by virtue of increasing the expression of major histocompatibility complex (MHC) antigens. IFNs are typically divided among three classes: Type I IFN, Type II IFN, and Type III IFN.

The tumor necrosis factor (TNF) superfamily is a protein superfamily of type II transmembrane proteins containing TNF homology domain and forming trimers. Members of this superfamily can be released from the cell membrane by extracellular proteolytic cleavage and function as a cytokine. These proteins are expressed predominantly by immune cells and regulate diverse cell functions, including regulation of immune response and inflammation, but also proliferation, differentiation, apoptosis and embryogenesis.

Interleukins (IL) are a type of cytokines that were first seen to be expressed by leukocytes with complex immunomodulatory functions—including cell proliferation, maturation, migration and adhesion, immune cell differentiation and activation, and inflammatory and anti- inflammatory actions. A few members act as chemoattractants for helper T cells, paralleling the actions of chemokines. Others are intimately involved in the cellular response to viral pathogens, making them akin to IFNs. Its are very important mediators of the physiological response to infection and also contribute significantly to the pathophysiology of a wide range of disorders.

In certain embodiments, the cytokine is an interleukin. In certain embodiments, the interleukin is selected from a group consisting of IL1β, IL1f9, IL10, IL27, IL33 and IL18BP.

The term “IL10” as used herein refers to interleukin 10, an anti-inflammatory cytokine also known as human cytokine synthesis inhibitory factor (CSIF). IL10 signals through a receptor complex consisting of two IL10 receptor-1 and two IL10 receptor-2 proteins. An exemplary complete cDNA sequence of human IL10 has the GENBANK accession number of AY029171.1 and an exemplary amino acid sequence of human IL10 has the GENBANK accession number of AAK38162.1. The term “IL10” encompasses homologs in other species, variants obtained by proteolytic processing, splice variants and allelic variants thereof.

The term “IL1β” as used herein refers to interleukin-1 beta, one of the interleukin-1 family, which are monokines produced by monocytes and macrophages, and corresponds to an inflammatory cytokine. It is a potent proinflammatory cytokine. Initially discovered as the major endogenous pyrogen, induces prostaglandin synthesis, neutrophil influx and activation, T-cell activation and cytokine production, B-cell activation and antibody production, and fibroblast proliferation and collagen production. Promotes Th17 differentiation of T-cells. Synergizes with IL12/interleukin-12 to induce IFNG synthesis from T-helper 1 (Thl) cells (Tominaga K. et al., Int. Immunol. 2000, 12: 151-160). An exemplary complete cDNA sequence of human IL10 has the GENBANK accession number of BC008678.1 and an exemplary amino acid sequence of human IL10 has the GENBANK accession number of AAH08678.1. The term “IL1β” encompasses homologs in other species, variants obtained by proteolytic processing, splice variants and allelic variants thereof.

The term “IL1F9” as used herein refers to interleukin-1 family member 9, also known as interleukin-36 gamma. It is a member of the interleukin-1 cytokine family. The activity of this cytokine is mediated via the interleukin-1 receptor-like 2 (IL1RL2/IL1R-rp2/IL-36 receptor), and is specifically inhibited by interleukin-36 receptor antagonist, (IL36RA/IL1F5/IL1delta). The expression of this cytokine in keratinocytes can also be induced by a multiple Pathogen-Associated Molecular Patterns (PAMPs). An exemplary complete cDNA sequence of human IL1F9 has the GENBANK accession number of BC096721.1 and an exemplary amino acid sequence of human IL1F9 has the GENBANK accession number of AAH96721.1. The term “IL1F9” encompasses homologs in other species, variants obtained by proteolytic processing, splice variants and allelic variants thereof.

The term “IL27” as used herein refers to Interleukin 27, a member of the IL-12 cytokine family. IL27 has pro- and anti-inflammatory properties, that can regulate T-helper cell development, suppress T-cell proliferation, stimulate cytotoxic T-cell activity, induce isotype switching in B-cells, and that has diverse effects on innate immune cells. An exemplary complete cDNA sequence of human IL27 has the GENBANK accession number of BC062422.1 and an exemplary amino acid sequence of human IL27 has the GENBANK accession number of AAH62422.1. The term “IL27” encompasses homologs in other species, variants obtained by proteolytic processing, splice variants and allelic variants thereof.

The term “IL33” as used herein refers to Interleukin 33, a member of the IL-1 family that potently drives production of T helper-2 (Th2)-associated cytokines. IL33 is a ligand for ST2 (IL1RL1), an IL-1 family receptor that is highly expressed on Th2 cells, mast cells and group 2 innate lymphocytes. An exemplary complete cDNA sequence of human IL33 has the GENBANK accession number of BC047085.1 and an exemplary amino acid sequence of human IL33 has the GENBANK accession number of AAH47085.1. The term “IL33” encompasses homologs in other species, variants obtained by proteolytic processing, splice variants and allelic variants thereof.

The term “IL18BP” as used herein refers to interleukin-18-binding protein. This protein binds to IL18, prevents the binding of IL18 to its receptor, and thus inhibits IL18-induced IFN-gamma production. An exemplary complete cDNA sequence of human IL18BP has the GENBANK accession number of BC044215.1 and an exemplary amino acid sequence of human IL18BP has the GENBANK accession number of AAH44215.1. The term “IL18BP” encompasses homologs in other species, variants obtained by proteolytic processing, splice variants and allelic variants thereof.

In certain embodiments, the cytokine is a chemokine. In certain embodiments, the chemokine comprises a CC-chemokine. In certain embodiments, the CC-chemokine is selected from a group consisting of CCL4, CCL8, CCL6, CCL9 and CCL11. In certain embodiments, the chemokine comprises a CXC-chemokine. In certain embodiments, the CXC-chemokine is selected from a group consisting of CXCL2, CXCL5, CXCL16, CXCL9 and CXCL13.

Chemokines are a family of small cytokines, or signaling proteins secreted by cells. Chemokines can be classified into four main subfamilies: CXC, CC, CX3C and XC, all of which exert their biological effects by interacting with G protein-linked transmembrane receptors called chemokine receptors that are selectively found on the surfaces of their target cells. . Chemokines are responsible for basal leukocyte migration (homeostatic) or actively participate in the inflammatory response attracting immune cells to the site of inflammation (inflammatory).

The term “CCL4” as used herein refers to C-C motif chemokine 4, a monokine with inflammatory and chemokinetic properties. An exemplary complete cDNA sequence of human CCL4 has the GENBANK accession number of BC107433.1 and an exemplary amino acid sequence of human CCL4 has the GENBANK accession number of AAI07434.1. The term “CCL4” encompasses homologs in other species, variants obtained by proteolytic processing, splice variants and allelic variants thereof.

The term “CCL6” as used herein refers to Chemokine (C-C motif) ligand 6, a small cytokine belonging to the CC chemokine family. An exemplary complete cDNA sequence of rat CCL6 has the GENBANK accession number of BC079460.1 and an exemplary amino acid sequence of rat CCL6 has the GENBANK accession number of AAH79460.1. Human counterparts homologous to the exemplified amino acid sequence can be found by BLAST, for example, NCBI accession numbers: NP 665905.2 and NP 116741.2. The term “CCL6” encompasses homologs in other species, variants obtained by proteolytic processing, splice variants and allelic variants thereof.

The term “CCL8” as used herein refers to chemokine (C—C motif) ligand 8, a small cytokine belonging to the CC chemokine family. It attracts monocytes, lymphocytes, basophils and eosinophils, and may play a role in neoplasia and inflammatory host responses. An exemplary complete cDNA sequence of human CCL8 has the GENBANK accession number of BC126242.1 and an exemplary amino acid sequence of human CCL8 has the GENBANK accession number of AAI26243.1. The term “CCL8” encompasses homologs in other species, variants obtained by proteolytic processing, splice variants and allelic variants thereof.

The Term “CCL9” as used herein refers to C-C motif chemokine 9, a monokine with inflammatory, pyrogenic and chemokinetic properties. It circulates at high concentrations in the blood of healthy animals. It binds to a high-affinity receptor activates calcium release in neutrophils. It also inhibits colony formation of bone marrow myeloid immature progenitors. An exemplary complete cDNA sequence of mouse CCL9 has the GI number of 85540457 and an exemplary amino acid sequence of mouse CCL9 has the NCBI accession number of NP 035468.1. Human counterparts homologous to the exemplified amino acid sequence can be found by BLAST, for example, NCBI accession numbers: NP 665905.2 and NP 005055.3. The term “CCL9” encompasses homologs in other species, variants obtained by proteolytic processing, splice variants and allelic variants thereof.

The term “CCL11” as used herein refers to C—C motif chemokine 11, a small cytokine belonging to the CC chemokine family. CCL11 selectively recruits eosinophils by inducing their chemotaxis, and therefore, is implicated in allergic responses. An exemplary complete cDNA sequence of human CCL11 has the GENBANK accession number of BC017850.1 and an exemplary amino acid sequence of human CCL11 has the GENBANK accession number of AAH17850.1. The term “CCL11” encompasses homologs in other species, variants obtained by proteolytic processing, splice variants and allelic variants thereof.

The term “CXCL2” as used herein refers to C-X-C motif chemokine 2, a hematoregulatory chemokine, which in vitro suppresses hematopoietic progenitor cell proliferation. An exemplary complete cDNA sequence of human CXCL2 has the GENBANK accession number of BC015753.1 and an exemplary amino acid sequence of human CXCL2 has the GENBANK accession number of AAH15753.1. The term “CXCL2” encompasses homologs in other species, variants obtained by proteolytic processing, splice variants and allelic variants thereof.

The term “CXCLS” as used herein refers to the C-X-C motif chemokine 5, a small cytokine belonging to the CXC chemokine family. It is produced following stimulation of cells with the inflammatory cytokines interleukin-1 or tumor necrosis factor-alpha. An exemplary complete cDNA sequence of human CXCLS has the GENBANK accession number of BC008376.1 and an exemplary amino acid sequence of human CXCLS has the GENBANK accession number of AAH08376.1. The term “CXCLS” encompasses homologs in other species, variants obtained by proteolytic processing, splice variants and allelic variants thereof.

The term “CXCL9” as used herein refers to chemokine (C—X—C motif) ligand 9, a small cytokine belonging to the CXC chemokine family that is also known as Monokine induced by gamma interferon (MIG). CXCL9 is a T-cell chemoattractant, which is induced by IFN-γ. An exemplary complete cDNA sequence of human CXCL9 has the GENBANK accession number of BC063122.1 and an exemplary amino acid sequence of human CXCL9 has the GENBANK accession number of AAH63122.1. The term “CXCL9” encompasses homologs in other species, variants obtained by proteolytic processing, splice variants and allelic variants thereof.

The term “CXCL13” as used herein refers to chemokine (C—X—C motif) ligand 13, a small cytokine belonging to the CXC chemokine family that is also known as B lymphocyte chemoattractant (BLC) or B cell-attracting chemokine 1 (BCA-1), is a protein ligand that in humans is encoded by the CXCL13 gene. CXCL13 is selectively chemotactic for B cells belonging to both the B-1 and B-2 subsets, and elicits its effects by interacting with chemokine receptor CXCR5. CXCL13 and its receptor CXCR5 control the organization of B cells within follicles of lymphoid tissues and is expressed highly in the liver, spleen, lymph nodes, and gut of humans. The gene for CXCL13 is located on human chromosome 4 in a cluster of other CXC chemokines. In T cells, CXCL13 expression is thought to reflect a germinal center origin of the T cell, particularly a subset of T cells called T follicular helper cells (or TFH cells). An exemplary complete CDNA sequence of human CXCL13 has the GENBANK accession number of EF064743.1 and an exemplary amino acid sequence of human CXCL13 precursor has the GENBANK accession number of AAH12589.1. The term “CXCL13” encompasses homologs in other species, variants obtained by proteolytic processing, splice variants and allelic variants thereof.

The term “CXCL16” as used herein refers to chemokine (C—X—C motif) ligand 16, a small cytokine belonging to the CXC chemokine family. CXCL16 is composed of a CXC chemokine domain, a mucin-like stalk, a transmembrane domain and a cytoplasmic tail containing a potential tyrosine phosphorylation site that may bind SH2. These are unusual features for a chemokine, and allow CXCL16 to be expressed as a cell surface bound molecule, as well as a soluble chemokine. CXCL16 is usually produced by dendritic cells found in the T cell zones of lymphoid organs, and by cells found in the red pulp of the spleen. Cells that bind and migrate in response to CXCL16 include several subsets of T cells, and natural killer T (NKT) cells. CXCL16 interacts with the chemokine receptor CXCR6, also known as Bonzo. An exemplary mRNA sequence of human CXCL16 has the GENBANK accession number of AF337812.1 and an exemplary amino acid sequence of human CXCL16 precursor has the GENBANK accession number of AAK38275.1. The term “CXCL16” encompasses homologs in other species, variants obtained by proteolytic processing, splice variants and allelic variants thereof.

Cell-adhesion molecules (CAMs) are a subset of cell adhesion proteins located on the cell surface involved in binding with other cells or with the extracellular matrix (ECM) in the process called cell adhesion. Cell adhesion molecules help cells stick to each other and to their surroundings. CAMs include the immunoglobulin super family of cell adhesion molecules (IgCAMs), Cadherins, Integrins, and the Superfamily of C-type of lectin-like domains proteins (CTLDs). In certain embodiments, Cell-adhesion molecules comprises ICAM1, CSF3r, Itgam, Siglecf, Adam8, Chl 1, Sirpa, Nrcam, Emilin2, Emilinl, Tubb6, and Parvb.

The term “ITGAM” as used herein refers to integrin alpha-M, implicated in various adhesive interactions of monocytes, macrophages and granulocytes as well as in mediating the uptake of complement-coated particles. An exemplary complete cDNA sequence of human ITGAM has the GENBANK accession number of BC096346.3 and an exemplary amino acid sequence of human ITGAM has the GENBANK accession number of AAH96346.1. The term “ITGAM” encompasses homologs in other species, variants obtained by proteolytic processing, splice variants and allelic variants thereof.

The term “SIGLECF” as used herein refers to sialic acid-binding Ig-like lectin F, also known as Siglec5, a cell surface protein that binds sialic acid. It is found primarily on the surface of immune cells and are a subset of the I-type lectins. An exemplary complete cDNA sequence of mouse SIGLECF has the GENBANK accession number of BC145038.1 and an exemplary amino acid sequence of mouse SIGLECF has the GENBANK accession number of AAI45039.1. Human counterparts homologous to the exemplified amino acid sequence can be found by BLAST, for example, NCBI accession numbers: NP_003821.1 and XP_016882908.1. The term “SIGLECF” encompasses homologs in other species, variants obtained by proteolytic processing, splice variants and allelic variants thereof.

The term “ADAM8” as used herein refers to disintegrin and metalloproteinase domain-containing protein 8, a member of the ADAM (a disintegrin and metalloproteinase domain) family. Members of this family are membrane-anchored proteins structurally related to snake venom disintegrins, and have been implicated in a variety of biological processes involving cell-cell and cell-matrix interactions, including fertilization, muscle development, and neurogenesis. The protein encoded by this gene may be involved in cell adhesion during neurodegeneration. An exemplary complete cDNA sequence of human ADAM8 has the GENBANK accession number of D26579.1 and an exemplary amino acid sequence of human ADAM8 has the GENBANK accession number of BAA05626.1. The term “ADAM8” encompasses homologs in other species, variants obtained by proteolytic processing, splice variants and allelic variants thereof.

The term “CHL1” as used herein refers to neural cell adhesion molecule L1-like protein, an extracellular matrix and cell adhesion protein that plays a role in nervous system development and in synaptic plasticity. Both soluble and membranous forms promote neurite outgrowth of cerebellar and hippocampal neurons and suppress neuronal cell death. An exemplary complete cDNA sequence of human CHL1 has the GENBANK accession number of BC104918.1 and an exemplary amino acid sequence of human CHL1 has the GENBANK accession number of AAI04919.1. The term “CHL1” encompasses homologs in other species, variants obtained by proteolytic processing, splice variants and allelic variants thereof.

The term “SIRPA” as used herein refers to signal-regulatory protein alpha, also known as tyrosine-protein phosphatase non-receptor type substrate 1, an immunoglobulin-like cell surface receptor for CD47. It acts as docking protein and induces translocation of PTPN6, PTPN11 and other binding partners from the cytosol to the plasma membrane. Supports adhesion of cerebellar neurons, neurite outgrowth and glial cell attachment. An exemplary complete cDNA sequence of human SIRPA has the GENBANK accession number of BC026692.1 and an exemplary amino acid sequence of human SIRPA has the GENBANK accession number of AAH26692.1. The term “SIRPA” encompasses homologs in other species, variants obtained by proteolytic processing, splice variants and allelic variants thereof.

The term “NRCAM” as used herein refers to neuronal cell adhesion molecule, a homophilic binding glycoprotein expressed on the surface of neurons, glia and skeletal muscle. It is required for normal responses to cell-cell contacts in brain and in the peripheral nervous system. Cell adhesion molecules (CAMs) are members of the immunoglobulin superfamily. An exemplary complete cDNA sequence of human NRCAM has the GENBANK accession number of BC115736.1 and an exemplary amino acid sequence of human NRCAM has the GENBANK accession number of AAI15737.1. The term “NRCAM” encompasses homologs in other species, variants obtained by proteolytic processing, splice variants and allelic variants thereof.

The term “EMILIN2” as used herein refers to elastin microfibril interfacer 2, a member of the EMILIN family of extracellular matrix glycoproteins. It has cell adhesive capacity. An exemplary complete cDNA sequence of human EMILIN2 has the GENBANK accession number of BC136541.1 and an exemplary amino acid sequence of human EMILIN2 has the GENBANK accession number of AAI36542.1. The term “EMILIN2” encompasses homologs in other species, variants obtained by proteolytic processing, splice variants and allelic variants thereof.

The term “EMILIN1” as used herein refers to elastin microfibril interfacer 1, a member of the EMILIN family of extracellular matrix glycoproteins. It has cell adhesive capacity. An exemplary complete cDNA sequence of human EMILIN1 has the GENBANK accession number of BC136279.1 and an exemplary amino acid sequence of human EMILIN1 has the GENBANK accession number of AAI36280.1. The term “EMILIN1” encompasses homologs in other species, variants obtained by proteolytic processing, splice variants and allelic variants thereof.

The term “TUBB6” as used herein refers to tubulin beta-6 chain, the major constituent of microtubules. It binds two moles of GTP, one at an exchangeable site on the beta chain and one at a non-exchangeable site on the alpha chain. An exemplary complete cDNA sequence of human TUBB6 has the GENBANK accession number of BC002654.1 and an exemplary amino acid sequence of human TUBB6 has the GENBANK accession number of AAH02654.1. The term “TUBB6” encompasses homologs in other species, variants obtained by proteolytic processing, splice variants and allelic variants thereof

The term “PARVB” as used herein refers to beta-parvin, an adapter protein that plays a role in integrin signaling via ILK and in activation of the GTPases CDC42 and RAC1 by guanine exchange factors. It is involved in the reorganization of the actin cytoskeleton and formation of lamellipodia. It plays a role in cell adhesion, cell spreading, establishment or maintenance of cell polarity, and cell migration. An exemplary complete cDNA sequence of mouse PARVB has the GENBANK accession number of AF237770.1 and an exemplary amino acid sequence of mouse PARVB has the GENBANK accession number of AAG27172.1. Human counterparts homologous to the exemplified amino acid sequence can be found by BLAST, for example, NCBI accession numbers: NP_001003828.1 and NP_037459.2. The term “PARVB” encompasses homologs in other species, variants obtained by proteolytic processing, splice variants and allelic variants thereof.

The term “ICAM1” as used herein refers to intercellular adhesion molecule 1, a cell surface glycoprotein which is typically expressed on endothelial cells and cells of the immune system. It binds to integrins of type CD11a/CD18, or CD1 1b/CD18 and is also exploited by rhinovirus as a receptor for entry into respiratory epithelium. An exemplary complete cDNA sequence of human ICAM1 has the GENBANK accession number of BC015969.2 and an exemplary amino acid sequence of human ICAM1 has the GENBANK accession number of AAH15969.1. The term “ICAM1” encompasses homologs in other species, variants obtained by proteolytic processing, splice variants and allelic variants thereof.

The term “CSF3R” as used herein refers to colony stimulating factor 3 Receptor, a receptor for granulocyte colony-stimulating factor (CSF3), essential for granulocytic maturation. It plays a crucial role in the proliferation, differentiation and survival of cells along the neutrophilic lineage. In addition it may function in some adhesion or recognition events at the cell surface. An exemplary complete cDNA sequence of human CSF3R has the GENBANK accession number of BC053585.1 and an exemplary amino acid sequence of human CSF3R has the GENBANK accession number of AAH53585.1. The term “CSF3R” encompasses homologs in other species, variants obtained by proteolytic processing, splice variants and allelic variants thereof.

The term “IL2” as used herein refers to interleukin-2, a type of cytokine signaling molecule in the immune system. It is a protein that regulates the activities of white blood cells (leukocytes, often lymphocytes, such as B cells) that are responsible for immunity. IL2 mediates its effects by binding to IL2 receptors, which are expressed by lymphocytes. IL2 is reported to induce proliferation of T cells (Lan, et al., Journal of Autoimmunity, 2008, 31(1):7-12), B cells (Karray, et al., J Exp Med. 1988 Jul. 1; 168(1): 85-94) and dendritic cells. An exemplary complete cDNA sequence of human IL2 has the GENBANK accession number of AH002842.2 and an exemplary amino acid sequence of human IL2 has the GENBANK accession number of AAD48509.1. The term “IL2” encompasses homologs in other species, variants obtained by proteolytic processing, splice variants and allelic variants thereof.

The term “IL21” as used herein refers to interleukin-21, which is also a cytokine that costimulates T and natural killer (NK) cell proliferation and function and regulates B cell survival and differentiation and the function of dendritic cells (see Croce et al., J Immunol Res. 2015; 2015: 696578). An exemplary complete cDNA sequence of human IL21 has the GENBANK accession number of NM_021803.3 and an exemplary amino acid sequence of human IL21 has the GENBANK accession number of NP 068575.1. The term “IL21” encompasses homologs in other species, variants obtained by proteolytic processing, splice variants and allelic variants thereof.

“Inducible T cell co-stimulator (ICOS)” is also known as “AILIM,” “CD278,” and “MGC39850”. An exemplary complete cDNA sequence of ICOS has the GENBANK accession number of NM_012092.3 and an exemplary amino acid sequence of human ICOS has GENBANK accession number of NP_036224. ICOS belongs to the CD28 and CTLA-4 cell-surface receptor family and is homologous to CD28 and CTLA-4. It forms homodimers by disulfide linkage and plays an important role in cell-cell signaling, immune responses, and regulation of cell proliferation during the formation of germinal centers, TB cell collaboration, and immunoglobulin class switching, via the PI3K and AKT pathways. Along with CD28 and CTLA-4, ICOS is expressed on activated CD4 and CD8 T cells and has potential role in regulating the adaptive T cell response, e.g. T cell activation and proliferation. Unlike CD28, which is constitutively expressed on T cells and provides co-stimulatory signals necessary for full activation of resting T cells, ICOS is expressed only after initial T cell activation. ICOS also plays a role in the development and function of other T cell subsets, including Th1, Th2, and Th17. ICOS co-stimulates T cell proliferation and cytokine secretion associated with both Thl and Th2 cells. ICOS knockout (KO) mice exhibit impaired development of autoimmune phenotypes in a variety of disease models, including diabetes (Th1), airway inflammation (Th2) and EAE neuro-inflammatory models (Th17). In addition to its role in modulating T effector (Teff) cell function, ICOS also modulates T regulatory cells (Tregs). Furthermore, ICOS is expressed at high levels on Tregs, and involves in Treg homeostasis and function (see US patent application US20160304610). The role of ICOS in promoting CD4+T cell proliferation is implicated to be independent of IL-2 signaling (see Wikenheiser DJ and Stumhofer JS, ICOS Co-Stimulation: Friend or Foe? Front Immunol. 2016; 7:304).

Agonist of ICOS (such as ICOSL) binds to the extracellular domain of ICOS, activates the ICOS signaling and thus increases the T cell activation and proliferation.

The term “ICOS ligand (ICOSL)” as used herein is also called “B7H2,” “GL50,” “B7-H2,” “B7RP1,” “CD275,” “ICOSLG,” “LICOS,” “B7RP-1,” “ICOS-L”, and “KIAA0653”, a co-stimulatory molecule of the B7 superfamily, functions as a positive signal in immune response. An exemplary complete cDNA sequence of ICOSL has the GENBANK accession number of NM_015259.5 and an exemplary amino acid sequence of human ICOSL has the GENBANK accession number of NP_056074.1. ICOSL shares 19-20% sequence identity with CD80/CD86 and is secreted or expressed as a cell surface protein. Human ICOSL has two splice variants (hGL50 and B7-H2/B7RP-1/hLICOS), both of which have identical extracellular domain but differ at the carboxyl-terminal of their cytoplasmic regions. In human, ICOSL is expressed on B cells, dendritic cells, monocytes/macrophages, and T cells. Unlike CD80/CD86, ICOSL does not interact with CD28 or CTLA-4 (CD152) but functions as a non-covalently linked homodimer on the cell surface and binds to ICOS. Human ICOSL is reported to bind to human CD28 and CTLA-4 (see US patent application US20160304610).

ICOS/ICOS-L's interaction is involved in T cell-mediated immune responses in vivo. Furthermore, in vivo deficiency in ICOS causes impaired germinal center (GC) formation (reduction in the numbers and size of the GCs), defect in isotype class switching in T cell-dependent B cell responses and defects in IL-4 and IL-13 production (see Khayyamian et al., ICOS-ligand, expressed on human endothelial cells, costimulates Thl and Th2 cytokine secretion by memory CD4 T cells, PNAS, Vol.9, No.9, 2002, 6198-6203). In the GC, long-lived plasma cells (LLPCs) and memory B cells (MBCs) undergo class-switching and somatic hypermutation to increase antibody affinity.

In certain embodiments, cultivating PBMCs in the presence of ICOS or ICOSL can enhance the total amount of antibody or antigen-binding fragment thereof produced by the PBMCs.

Agonist of ICOS can be screened by determination of their affinity and specificity of binding. The method for determining the affinity and specificity of binding, such as competitive and non-competitive binding assay are known in the art, including ELISA, RIA, flow cytometry, etc. The effects of ICOS agonists can be determined by a functional assay detecting the T cell activation by ICOS. The T cell activation can be measured via detection of CD4+ T cell proliferation, cell cycle progression, release of cytokines, such as IL-2, upregulation of CD25 and CD69, etc.

The ICOS agonists include compounds or proteins, such as an agonist antibody JTX-2011 (Jounce Therapeutics Inc) and GSK3359609 (GSK), and the antibodies described in US patent application US20160304610, US 20170174767, as well as WO 2012/131004.

CD40L, as used herein, is also called CD40 ligand or CD154, a protein that is primarily expressed on activated T cells (its expression has since been found on a wide variety of cells, including platelets, mast cells, macrophages, basophils, NK cells, B lymphocytes, as well as non-hematopoietic cells) and is a member of the TNF superfamily of molecules. It binds to CD40 on antigen-presenting cells (APC) and acts as a costimulatory molecule that is particularly important on a subset of T cells called T follicular helper cells (TFH cells). On TFH cells, CD40L promotes B cell maturation and function by engaging CD40 on the B cell surface and therefore facilitating cell-cell communication. An exemplary complete cDNA sequence of CD40L has the GENBANK accession number of NM_000074.2 and an exemplary amino acid sequence of human CD40L has the GENBANK accession number of NP_000065.1.

The phrase “B-cell activating factor” or “BAFF” or “Baff” as used herein refers to a tumor necrosis family ligand, e.g., a TNF family ligand. BAFF is expressed on the surface of a cell and serves as a regulatory protein involved in interactions between membrane surface proteins on immune cells, e.g., B cells. Secreting BAFF is efficient B cell growth factor, and help B cell to proliferate and function as a co-stimulator. It is reported that BAFF is critical to the survival of antibody-secreting cell from memory cells (Avery DV et al., J Clin Invest, 2003, 112:286-97). An exemplary amino acid sequence of human CD40L has the GENBANK accession number of AAD25356.1.

“OX40L” is the ligand for OX40 (CD134) and is expressed on cells such as DC2s (a subtype of dendritic cells) enabling amplification of Th2 cell differentiation. OX40L has also been designated CD252 (cluster of differentiation 252). It has been reported that OX40 co-express with ICOS in T follicular helper cells (Tfh) and affect interaction between Tfh cells-B cells in germinal center (GC), thereby affecting the B cell development and differentiation and maturation of plasma cells in the GC. An exemplary cDNA sequence of human OX40 has the GENBANK accession number of AJ277151.1 and an exemplary amino acid sequence of human OX40L has the GENBANK accession number of CAB96543.1.

The term “Toll-like receptor (TLR)” is a family of proteins that play a key role in the innate immune system (non-specific immunity). They are single, membrane-spanning, non-catalytic receptors usually expressed on sentinel cells such as macrophages and dendritic cells, that recognize structurally conserved molecules derived from microbes. Beside the extracellular and transmembrane domain, a TLR comprises a cytoplasmic Toll-interleukin) receptor-resistance (TIR) domain. Once these microbes have breached physical barriers such as the skin or intestinal tract mucosa, they are recognized by TLRs, which activate immune cell responses. The TLRs recognize highly conserved structural motifs, i.e. pathogen-associated molecular patterns (PAMPs) that are exclusively expressed by microbial pathogens, such as lipopolysaccharide (LPS) from gram-negative bacteria and lipoteichoic acid (LTA) from gram-positive bacteria and flagellin, etc, or danger-associated molecular patterns (DAMPS) that are endogenous molecules released from necrotic or dying cells. Many tumor cells undergo necrosis mediated by the immune system and may lead to further activation of an inflammation response via TLRs. The human TLR family includes TLR1, TLR2, TLR3, TLR4, TLRS, TLR6, TLR7, TLR8, TLR9, and TLR10, which are expressed on a variety of immune cell types. Mouse TLR family includes TLR1-9 and TLR 11-13.

“Toll-like receptor ligand” as used herein refers to agonists or antagonists of Toll-like receptor. In certain embodiments, the TLR ligand is an agonist, such as pathogen-associated molecular patterns (PAMPs). Examples of the TLR agonist that activates TLR includes, but not limited to imiquimod, GS-9620 (Gilead, see Roethle et al, 2013), compound 32 (GSK2245035, GSK, see Biggadike et al, 2016), and resiquimod (R848), imidazoquinolines, nucleic acids comprising an CpG ODN, such as an unmethylated CpG dinucleotide (e.g. ODN2216) and poly I:C, monophosphoryl lipid A (MPLA) or other lipopolysaccharide derivatives, single-stranded or double-stranded RNA, flagellin, muramyl dipeptide, TSLP, Tumor necrosis factor (TNF) alpha, type I Interferons (e.g. IFN α/β), type II interferon (e.g. IFNγ), lipids, avasimid, EFNB1, EPHB4, Plexin B2, semaphoring 4C, BLIMP-1, and IRF4. (see J. Med. Chem. Roethle et al, 2013. Identification and Optimization of Pteridinone Toll-like Receptor 7 (TLR7) Agonists for the Oral Treatment of Viral Hepatitis. J. Med. Chem. Biggadike et al, 2016. 59,1711-1726. Discovery of 6-Amino-2-{[(1S)- 1-methylbutyl]oxy}-9-[5-(1-piperidinyl)pentyl]-7,9-dihydro⁻ 8H⁻ purin-8-one (GSK2245035), a Highly Potent and Selective Intranasal Toll-Like Receptor 7 Agonist for the Treatment of Asthma.)

TLR agonists specific to the TLR types are reported, for example, BCG (TLR1, 2, 4, and 6), lipopeptides (TLR1, 2, and 6), monophosphoryl lipid A (MPL), LPS, RC529, AS01, AS02, AS04 and glucopyranosyl lipid adjuvant (GLA-SE) (TLR4), poly(I:C) (TLR3), flagellin (TLRS), single stranded and R484/resiquimod (TLR7 and TLR8) or double stranded (ds) RNA (TLR3), imiquimod and Type 1 interferon (TLR7) and DNA containing the CpG motif, AS15, and IC31 (TLR9). Endogenous molecules released from stressed or dead cells such as heat shock proteins (HSP; TLR2 and TLR4) and high mobility group box 1 (HIMGB1; TLR2 and TLR4) are also reported important TLR agonists (see Deng Sl et al., Recent advances in the role of toll-like receptors and TLR agonists in immunotherapy for human glioma, (see Protein Cell 2014, 5(12):899-911; Zhang W W and Matlashewski G, Immunization with a Toll-Like Receptor 7 and/or 8 Agonist Vaccine Adjuvant Increases Protective Immunity against Leishmania major in BALB/c Mice, INFECTION AND IMMUNITY, August 2008, p. 3777-3783; Gauwelaert N D et al., The TLR4 Agonist Vaccine Adjuvant, GLA-SE, Requires Canonical and Atypical Mechanisms of Action for TH1 Induction, PLoS One. 2016 Jan. 5; 11(1):e0146372; Maisonneuve C et al., Unleashing the potential of NOD- and Toll-like agonists as vaccine adjuvants. Proc Natl Acad Sci U S A. 2014 Aug. 26; 111(34): 12294-9).

In certain embodiments, the antibody-enhancing composition is added to the medium at the beginning of the cultivation, or 1, 2, 3, 4, 5, 6, 7, 8, 9, 10, 11, 12, 13, 14, 15, 16, 17, 18, 19, 20, 21 days later. In certain embodiments, the composition is removed from the medium 1, 2, 3, 4, 5, 6, 7, 8, 9, 10, 11, 12, 13, 14, 15, 16, 17, 18, 19, 20, 21 days later.

In certain embodiments, two or more of the antibody-enhancing factors of the antibody-enhancing composition exhibit synergistic effects on stimulating in vitro antibody production. The two or more antibody-enhancing factors can be selected from ADSPs, CD40L, ICOSL, ICOS, TLR agonists, co-stimulators (CD40, CD40L, ICOSL, ICOS, APRIL, B cell activating factor of the TNF family (BAFF), OX40, or OX40L), toll-like receptor (TLR) agonists (TLR1 agonist, TLR2 agonist, TLR3 agonist, TLR4 agonist, TLRS agonist, TLR6 agonist, TLR7 agonist, TLR8 agonist and TLR9 agonist), CpG oligodeoxynucleotides (CpG2006, or D/K CpG), anti-apoptotic proteins (Bcl-2, Bcl-6, Bcl-XL, Bcl-w, Mcl-1, or an analog thereof), TNFs, type I Interferons (e.g. IFN-α, IFN-β, IFN-ε, IFN-κ and IFN-ω), type II interferon (e.g. IFN γ), lipids, avasimid, EFNB1, EPHB4, Plexin B2, Semaphorin 4C, BLIMP-1, and IRF4.

In Vitro Antibody Production

The present disclosure provides a method for producing an antibody or an antigen-binding fragment thereof specifically binding to an antigen of interest, the method comprising

mixing the antigen, an antibody-generating cell composition (AGC), and an antibody-enhancing composition in a medium to form a mixture,

cultivating the mixture,

obtaining the antibody from the mixture,

wherein the AGC comprises at least one B cell and at least one additional type of cell derived from peripheral blood mononuclear cells (PBMCs), and the antibody-enhancing composition comprises one or more adipose tissue-derived secretory proteins (ADSPs). In certain embodiments, the antibody-enhancing composition further comprises IL2 and/or IL21.

In certain embodiments, the B cell prior to the cultivation is non-mature. In certain embodiments, the B cell prior to the cultivation does not produce an antibody to the antigen of interest.

The present disclosure also provides a method for producing an antibody or antigen-binding fragment thereof specifically binding to an antigen of interest, the method comprising:

mixing the antigen, an antibody-generating cell composition (AGC), and an antibody-enhancing composition in a medium to form a mixture,

cultivating the mixture,

obtaining the antibody from the mixture,

wherein the AGC comprises at least one B cell and at least one additional type of cell derived from peripheral blood mononuclear cells (PBMCs), and the antibody-enhancing composition comprises IL2, IL21, and one or more adipose tissue-derived secretory proteins (ADSPs).

The ADSP, IL2 and IL21 can be introduced to the medium together at one time or separately in any suitable order. IL-2 and/or IL21 is capable of promoting proliferation of the B cell, and ADSP and the antigen are capable of stimulating the B cells to generate antibody, and IL21 is also capable of promoting class switch of the antibodies from IgM to IgG. In certain embodiments, IL2 is first added to the medium, followed by the antigen and the ADSP, and then IL21.

In certain embodiments, addition of the ADSP to the medium generate antibodies with antigen-specificity. In certain embodiments, the ADSP includes cytokines, e.g. IL1β, IL1f9, IL10, IL27, IL33, IL18BP, chemokines, e.g. CCL8, CCL4, CXCL2, CCL6, CCL9, CCL11, CXCL5, CXCL2, and CXCL9, and cell-adhesion molecules, e.g. ICAM1, CSF3r, Itgam, Siglecf, Adam8, Chl 1, Sirpa, Nrcam, Emilin2, Emilinl, Tubb6, and Parvb. In certain embodiments, the ADSP comprises IL1(3, CCL8, and CXCL5. In certain embodiments, the antibodies or antigen-binding fragments generated according to the methods provided herein specifically bind human and/or non-human antigen with a binding affinity (K_{D}) of about 0.01 nM to about 100 nM, about 0.1 nM to about 100 nM, 0.01 nM to about 10 nM, about 0.1 nM to about 10 nM, 0.01 nM to about 5 nM, about 0.1 nM to about 5 nM, 0.01 nM to about 1 nM, about 0.1 nM to about 1 nM or about 0.01 nM to about 0.1 nM).

A first group of ADSPs can be added to the medium for a first period of time after the beginning of the cultivation, followed by addition to the medium a second group of ADSPs for a second period of time. In certain embodiments, said first group of ADSPs are removed before addition of the second group of ADSPs. In certain embodiments, said second group of ADSPs are removed from the mixture at the end of the second period of time. In certain embodiments, said “first period” or “second period” refers to, e.g. 0 hour, 0.5 hour, 1 hour, 2 hours, 3 hours, 6 hours, 12 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, one month or longer. In certain embodiments, the “first period” or “second period” are of the same or different length of time (or time span). In certain embodiments, the first and/or second group of ADSPs are present in the mixture 0 hour, 0.5 hour, 1 hour, 2 hours, 3 hours, 6 hours, 12 hours, 1 day, 2 days, 3 days, 4 days, 5 days, 6 days, 7 days, 8 days, 9 days, 10 days after the beginning of the cultivation. In certain embodiments, the first batch and second group of ADSPs are the same or different.

In certain embodiments, the first or second group of ADSPs includes cytokines, e.g. IL1β, IL1f9, 11,10, IL27, IL33, IL18BP, chemokines, e.g. CCL8, CCL4, CXCL2, CCL6, CCL9, CCL11, CXCL5, CXCL2, and CXCL9, and cell-adhesion molecules, e.g. ICAM1, CSF3r, Itgam, Siglecf, Adam8, Chl1, Sirpa, Nrcam, Emilin2, Emilin1, Tubb6, and Parvb.

The ADSPs are present in the mixture at a concentration of at least 0.5, 1, 2, 3, 4, 5, 6, 7, 8, 9, 10, 12, 15, 20, 25, 30, 35, 40, 45, 50, 55, 60, 65, 70, 75, 80, 85, 90, 95, 100, 150, 200, 250, 300, 350, 400, 450, 500, 1000, 1500, 2000, 3000, 4000, 5000 or more ng/ml, or 0.5, 1, 2, 3, 4, 5, 6, 7, 8, 9, 10, 12, 15, 20, 25, 30, 35, 40, 45, 50, 55, 60, 65, 70, 75, 80, 85, 90, 95, 100, 150, 200, 250, 300, 350, 400, 450, 500, 1000, 1500, 2000, 3000, 4000, 5000 or more pg/ml, or 0.1, 0.2, 0.5, 1, 2, 3, 4, 5, 6, 7, 8, 9, 10, 12, 15, 20, 24, 25, 28, 30, 35, 40, 45, 50, 55, 60, 65, 70, 75, 80, 85, 90, 95, 100, 150, 200, 250, 300, 350, 400, 450, 500, 550, 600, 700, 800, 900, 1000 or more nM.

In certain embodiments, the IL1β is present in the mixture at a concentration of 40, 400, or 1000 ng/ml. In certain embodiments, the CCL8 is present in the mixture at a concentration of 10, 50, or 100 ng/ml. In certain embodiments, the CXCL5 is present in the mixture at a concentration of 1, 10, or 20 ng/ml. In certain embodiments, the CCL4 is present in the mixture at a concentration of 5 ng/ml. In certain embodiments, the CXCL2 is present in the mixture at a concentration of 10 ng/ml. In certain embodiments, the CXCL16 is present in the mixture at a concentration of 2 ng/ml.

In certain embodiments, IL2 is present in the mixture for 0 hour, 0.5 hour, 1 hour, 2 hours, 3 hours, 6 hours, 12 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, one month or longer. In certain embodiments, 11,21 is present in the mixture for 0 hour, 0.5 hour, 1 hour, 2 hours, 3 hours, 6 hours, 12 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, one month or longer. In certain embodiments, IL2 and/or IL21 are present in the mixture 0 hour, 0.5 hour, 1 hour, 2 hours, 3 hours, 6 hours, 12 hours, 1 day, 2 days, 3 days, 4 days, 5 days, 6 days, 7 days, 8 days, 9 days, 10 days after the beginning of the cultivation.

In certain embodiments, the IL2 is present in the mixture at a concentration of at least 0.5, 1, 2, 3, 4, 5, 6, 7, 8, 9, 10, 12, 15, 20, 25, 30, 35, 40, 45, 50, 55, 60, 65, 70, 75, 80, 85, 90, 95, 100, 150, 200, 250, 300, 350, 400, 450, 500 or more ng/ml, or 0.5, 1, 2, 3, 4, 5, 6, 7, 8, 9, 10, 12, 15, 20, 25, 30, 35, 40, 45, 50, 55, 60, 65, 70, 75, 80, 85, 90, 95, 100, 150, 200, 250, 300, 350, 400, 450, 500 or more μg/ml, or 0.1, 0.2, 0.5, 1, 2, 3, 4, 5, 6, 7, 8, 9, 10, 12, 15, 20, 24, 25, 28, 30, 35, 40, 45, 50, 55, 60, 65, 70, 75, 80, 85, 90, 95, 100, 150, 200, 250, 300, 350, 400, 450, 500, 550, 600, 700, 800, 900, 1000 or more nM.

In certain embodiments, the IL21 is present in the mixture at a concentration of at least 0.5, 1, 2, 3, 4, 5, 6, 7, 8, 9, 10, 12, 15, 20, 25, 30, 35, 40, 45, 50, 55, 60, 65, 70, 75, 80, 85, 90, 95, 100, 150, 200, 250, 300, 350, 400, 450, 500, 600, 700, 800, 1000 or more ng/ml, or 0.5, 1, 2, 3, 4, 5, 6, 7, 8, 9, 10, 12, 15, 20, 25, 30, 35, 40, 45, 50, 55, 60, 65, 70, 75, 80, 85, 90, 95, 100, 150, 200, 250, 300, 350, 400, 450, 500 or more μg/ml, or 0.1, 0.2, 0.5, 1, 2, 3, 4, 5, 6, 7, 8, 9, 10, 12, 15, 20, 24, 25, 28, 30, 35, 40, 45, 50, 55, 60, 65, 70, 75, 80, 85, 90, 95, 100, 150, 200, 250, 300, 350, 400, 450, 500, 550, 600, 700, 800, 900, 1000 or more nM.

In certain embodiments, the IL2 and IL21 are present in the mixture with the concentration ata ratio of 1:1, 1:2, 1:5, 1:10, 1:20, 1:30, 1:40, 1:50, 1:60, 1:70, 1:80, 1:90, 1:100, 1:150, 1:200, 1:500, 1:1000, 1:2000, 1:5000, 1:10000, or 1:20000.

In certain embodiments, the concentration of IL2 present in the mixture is 10 ng/ml. In certain embodiments, the concentration of IL21 present in the mixture is 50 ng/ml.

In certain embodiments, the mixture further comprises an antigen. The antigen is present in the mixture at the beginning of the cultivation, or 1, 2, 3, 4, 5, 6, 7, 8, 9, 10, 11, 12, 13, 14, 15, 16, 17, 18, 19, 20, 21 days after the beginning of the cultivation. In certain embodiments, the antigen is present in the mixture for at least 0.5 day, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25, one month or longer.

The present disclosure provides methods for inducing proliferation of antibody-generating cell composition (AGC), B cell activation and differentiation, B cell maturation, and/or promoting class switch in an AGC to produce IgG, wherein the method comprising cultivating the AGC in a medium comprising IL2, an ADSP, and/or IL21. In certain embodiments, the AGC comprises PBMCs.

The AGCs or the PBMCs can be cultured in vitro according to known protocols in the art (Panda, S. K. and Ravindran, B. (2013). In vitro Culture of Human PBMCs. Bio protocol 3(3): e322.). Proliferation of the AGC or the PBMCs after stimulation can be measured using common methods in the art, such as flow cytometry.

B cell activation and differentiation is a process of B lymphocyte in periphery undergoes antigen-induced activation and differentiation. Activated B cells can give-rise to antibody-secreting plasma cells or memory B cells. The class switch occurs at the stage of plasma cells. B cells may first differentiate into a plasmablast-like cell, then differentiate into a plasma cell, which are generated later in an infection and, compared to plasmablasts, have antibodies with a higher affinity towards their target antigen due to affinity maturation in the germinal center (GC) and produce more antibodies (see Nutt et al., Nature Reviews Immunology. 2015, 15 (3): 160). Plasma cells typically result from the germinal center reaction from T cell-dependent (TD) activation of B cells, however they can also result from T cell-independent (TI) activation of B cells (see Bortnick et al., The Journal of Immunology. 188 (11): 5389-5396). B cell activation or differentiation can be detected or confirmed in vitro by methods known in the art, for example, by cell labelling with CD19, IgM, IgD, IgA antibodies and cell sorting using FACS. Memory B cells can be determined as CD19⁺IgM⁻IgA⁻IgD⁻, while IgG-producing B cells can be recognized as CD19⁺IgG⁺.

B cell development is the differentiation of lymphoid precursor cells differentiate into the earliest distinctive B -lineage cell (the progenitor B cell (pro-B cell)), which expresses a transmembrane tyrosine phosphatase, CD45R (or B220 in mice). Proliferation and differentiation of pro-B cells into precursor B cells (pre-B cells) requires the microenvironment provided by the bone marrow stromal cells, which interact directly with pro-B and pre-B cells, and secrete various cytokines, notably IL-7, that support the developmental process.

B cell maturation is a period which depends on rearrangement of the immunoglobulin DNA in the lymphoid stem cells. During B-cell development, sequential Ig-give rearrangements transform a pro-B cell into an immature B cell expressing mIgM with a single antigenic specificity. Future development yields mature naïve B cells, still of a single specificity, expressing both mIgM and mIgD. Only pre-B cells that are able to express membrane-bound μ heavy chains in association with surrogate light chains are able to proceed along the maturation pathway. Following the establishment of an effective pre-B cell receptor, each pre-B cell undergoes multiple cell divisions, perhaps six to eight, producing as many as 256 descendants. Each of these progeny pre-B cells may then rearrange different light-chain gene segments, thereby increasing the overall diversity of the antibody repertoire. In certain embodiments, the B cell maturation occurs in periphery. B cell maturation can be detected or confirmed in vitro by methods known in the art, for example, by detecting B cell surface markers, for example, immature B cells express mIgM and mIgD, and mature B cells express mIgG, mIgA and mIgD. Those skilled in the art will appreciate that methods such as cell staining and cell sorting with labeled antibodies against the above markers can be used.

Class switch is also referred to isotype switching, isotypic commutation or class-switch recombination (CSR). It is a biological mechanism that changes a B cell's production of immunoglobulin (antibodies) from one type to another, such as from the isotype IgM to the isotype IgG and IgE. During this process, the constant-region portion of the antibody heavy chain is changed, but the variable region of the heavy chain stays the same. Since the variable region does not change, class switching does not affect antigen specificity. Instead, the antibody retains affinity for the same antigens, but can interact with different effector molecules (see Honjo et al., Immunity, 1 Jun. 2004, 20(6):659-668). Methods for determination of IgG and IgM and the levels thereof are known in the art, for example, by ELISA using the antibodies specific for the isotypes.

In certain embodiments, the antibodies generated according to the method provided herein comprise IgG. In certain embodiments, the antibodies according to the method provided herein comprise increased percentage of IgG.

In certain embodiments, the method further comprises isolating the antibody from the mixture. For example, the method further comprises fusing the antibody-producing B cell with an immortal cell line to obtain a hybridoma. In certain embodiments, the method further comprises isolating the RNA of the antibody produced and cloning a library of single domain antibodies using molecular biology techniques known in the art and subsequent selection by using phage display

The term “hybridoma” used herein refers to a fused hybrid cell in the process of hybridoma technology, which is a method for producing large numbers of monoclonal antibodies. The antibody-producing B cells in response to an immune response are harvested and in turn fused with immortal B cell cancer cells, a myeloma, to produce a hybrid cell line called a hybridoma, which has both the antibody-producing ability of the B-cell and the exaggerated longevity and reproductivity of the myeloma. The hybridomas can be grown in culture, each culture starting with one viable hybridoma cell, producing cultures each of which consists of genetically identical hybridomas which produce one antibody per culture (monoclonal) rather than mixtures of different antibodies (polyclonal). In contrast to polyclonal antibodies, which are mixtures of many different antibody molecules, the monoclonal antibodies produced by each hybridoma line are all chemically identical.

The techniques for selecting “phage display libraries” refers to a method that repertoires of V_{H} and V_{L} genes are separately cloned by polymerase chain reaction (PCR) and recombined randomly in phage libraries, which can then be screened for antigen-binding phage as described in Winter, G. et al., Ann. Rev. Immunol. 12 (1994) 433-455. Phage typically display antibody fragments, either as single-chain Fv (scFv) fragments or as Fab fragments. Libraries from immunized sources (for example the antibody-producing PBMCs made by methods provided herein) provide high-affinity antibodies to the antigen without the requirement of constructing hybridomas. Alternatively, the naive repertoire can be cloned (e.g., from human) to provide a single source of antibodies to a wide range of non-self and also self antigens without any immunization as described by Griffiths, A. D. et al., EMBO J. 12 (1993) 725-734. Finally, naive libraries can also be made synthetically by cloning non-rearranged V-gene segments from stem cells, and using PCR primers containing random sequence to encode the highly variable CDR3 regions and to accomplish rearrangement in vitro, as described by Hoogenboom, H. R. and Winter, G., J. Mol. Biol. 227 (1992) 381-388. Patent publications describing human antibody phage libraries include, for example: U.S. Pat. No. 5,750,373, and US 2005/0079574, US 2005/0119455, US 2005/0266000, US 2007/0117126, US 2007/0160598, US 2007/0237764, US 2007/0292936, and US 2009/0002360. Similar display libraries includes ribosome display, yeast display, bacteria display, baculovirus display, mammal cell display, or mRNA display libraries (see, e.g., U.S. Pat. No. 7,244,592; Chao et al., Nature Protocols. 1:755-768, 2006). These display methods are all conventional techniques in the art, the specific operations thereof can be found in corresponding textbooks or operation manuals, see, e.g. Mondon P et al., Front. Biosci. 13:1117-1129, 2008.

In certain embodiment, the method further comprises obtaining a nucleic acid molecule encoding a variable region of the antibody generated from the mixture. In certain embodiment, the method further comprises introducing the nucleic acid molecule into a host cell under a condition suitable for expressing the antibody or the antigen-binding fragment thereof. In certain embodiments, the host cell is CHO cell.

The term “nucleic acid” or “polynucleotide” as used herein refers to deoxyribonucleic acids (DNA) or ribonucleic acids (RNA) and polymers thereof in either single- or double-stranded form. Unless otherwise indicated, a particular polynucleotide sequence also implicitly encompasses conservatively modified variants thereof (e.g. degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

Host cells are transformed with the nucleic acid sequence encoding a variable region of the antibody or the antigen-binding fragment thereof for antibody production and cultured in conventional nutrient media modified as appropriate for inducing promoters, selecting transformants, or amplifying the genes encoding the desired sequences. In another embodiment, the antibody or the antigen-binding fragment thereof may be produced by homologous recombination known in the art.

In certain embodiments, the method further comprises obtaining a nucleic acid sequence encoding a variable region of the antibody generated from the mixture.

DNA encoding the monoclonal antibody is readily isolated and sequenced using conventional procedures (e.g. by using oligonucleotide probes that are capable of binding specifically to genes encoding the heavy and light chains of the antibody). The encoding DNA may also be obtained by synthetic methods.

In certain embodiments, the method further comprises evaluating if the antibody or antigen-binding fragment thereof so produced specifically binds to an antigen of interest.

In certain embodiments, evaluation of the binding specificity is performed by immunoassays, such as ELISA, or fluoroimmunoassay.

In certain embodiments, evaluation of the binding specificity is determined by binding affinity. Binding affinity of an antibody or antigen-binding fragment thereof produced according to the method provided herein can be represented by K_{D} value. K_{D} used herein refers to the ratio of the dissociation rate to the association rate (kofilkon), which may be determined by using any conventional method known in the art, including but are not limited to surface plasmon resonance method, microscale thermophoresis method, HPLC-MS method and flow cytometry (such as FACS) method. In certain embodiments, the K_{D} value can be appropriately determined by using flow cytometry method. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Using Antibodies, A Laboratory Manual (1998), for a description of immunoassay formats and conditions that can be used to determine specific immunoreactivity). Typically a specific or selective binding reaction will produce a signal at least twice over the background signal and more typically at least 10 to 100 times over the background.

Alternatively, binding affinity of the antibodies and antigen-binding fragments thereof produced according to the method provided herein to a certain antigen can also be represented by “half maximal effective concentration” (EC₅₀) value, which refers to the concentration of an antibody where 50% of its maximal effect (e.g., binding) is observed. The EC₅₀ value can be measured by methods known in the art, for example, sandwich assay such as ELISA, Western Blot, flow cytometry assay, and other binding assay.

Compositions for In Vitro Immunization

The present disclosure provides herein compositions comprising an antibody-generating cell composition (AGC) comprising at least one B cell and at least one additional type of cell derived from peripheral blood mononuclear cells (PBMCs), an antibody-enhancing composition, and a medium.

In certain embodiments, the compositions further comprise an antigen of interest. In certain embodiments, the antibody-enhancing composition further comprises IL2 and/or IL21.

In certain embodiments, the AGC comprises PBMCs. In certain embodiments, the AGC comprises at least one B cell. In certain embodiments, the AGC comprises at least one of B cell, at least one T cell (e.g. T follicular helper cell), at least one dendritic cell, at least one NK cell, at least one monocyte, and at least one adipocyte.

For example, in certain embodiments, the AGC comprises at least one B cell and at least one T cell (e.g. T follicular helper cell). In certain embodiments, the AGC comprises at least one B cell and at least one dendritic cell. In certain embodiments, the AGC comprises at least one B cell, at least one T cell (e.g. T follicular helper cell), and at least one dendritic cell. In certain embodiments, the AGC comprises at least one B cell and at least one NK cell. In certain embodiments, the AGC comprises at least one B cell and at least one monocyte. In certain embodiments, the AGC comprises at least one B cell, T cell (e.g. T follicular helper cell), and at least one NK cell. In certain embodiments, the AGC comprises at least one B cell, at least one T cell (e.g. T follicular helper cell), at least one dendritic cell and at least one NK cell.

In certain embodiments, the AGC further comprises adipocytes. In certain embodiments, the AGC comprises at least one adipocyte and B cell. In certain embodiments, the AGC comprises at least one adipocyte, at least one B cell and at least one T cell (e.g. T follicular helper cell). In certain embodiments, the AGC comprises at least one adipocyte, at least one B cell, and at least one dendritic cell. In certain embodiments, the AGC comprises at least one adipocyte, B cell, T cell (e.g. T follicular helper cell), and at least one dendritic cell.

In certain embodiments, at least one of the B cells, T follicular helper cell, dendritic cell, and adipocyte is human cell. In certain embodiments, the B cell is human B cell. In certain embodiments, the PBMCs are derived from human PBMCs.

In certain embodiments, the PBMCs are isolated from a human donor. In certain embodiments, the PBMCs are derived from stem cells.

The isolated antibody-enhancing factors comprise the antibody-enhancing factors previously described in the present disclosure. In certain embodiments, the isolated antibody-enhancing factors comprise isolated ADSP, CD40L, ICOSL, ICOS, TLR agonist, which are previously described.

A medium contained in the composition provides a condition suitable for the antibody-generating cell composition (AGC) to express the antibody or the antigen-binding fragment thereof to an antigen of interest. The medium can be a solid, liquid or semi-solid designed to support the growth of microogranisms or cells that supplies the essential nutrients (amino acids, carbohydrates, vitamins, minerals), growth factors, hormones, and gases (CO₂, O₂), and regulates the physio-chemical environment (pH buffer, osmotic pressure, temperature) to the cells. Commercially available media such as Ham's F10 (Sigma), Minimal Essential Medium (MEM), (Sigma), RPMI-1640 (Sigma), and Dulbecco's Modified Eagle's Medium (DMEM), Sigma) are suitable for culturing the antibody-generating cell composition (AGC). In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30,985 may be used as culture media for the antibody-generating cell composition (AGC). Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with antibody-generating cell composition (AGC) selected for expression, and will be apparent to the ordinarily skilled artisan.

Identification of Antibody-enhancing Factor for In Vitro Immunization

The present disclosure provides herein a method for identifying an antibody-enhancing factor for in vitro immunization, comprising:
a) isolating total RNA from a cell derived from a lymph node of an animal immunized with an antigen of interest;
b) comparing the RNA levels of the total RNA isolated from the step a) with that of a control animal without immunization to determine a gene which encodes a protein and whose expression level is upregulated;
c) cultivating PBMCs in a medium comprising the antigen of interest, IL2, IL21 and the protein;
d) identifying the protein as an antibody-enhancing factor for in vitro immunization if the protein enhances antibody production.

In certain embodiments, the cell is an adipocyte, a T follicular helper cell, a B cell or a dendritic cell. In certain embodiments, the protein is expressed by the adipocyte, the T follicular helper cell, the B cell or the dendritic cell. In certain embodiments, the protein enhances IgG production.

Chimeric Antigen Receptor (CAR)

The term “chimeric antigen receptor” or “CAR” as used herein refers to an artificially constructed hybrid protein or polypeptide containing an antigen binding domain of an antibody (e.g., a single chain variable fragment (scFv)) linked to T-cell signaling or T-cell activation domains (see, e.g., Kershaw et al., supra, Eshhar et al., Proc. Natl. Acad. Sci. USA, 90(2): 720-724 (1993), and Sadelain et al., Curr. Opin. Immunol. 21(2): 215-223 (2009)). CARs are capable of redirecting T-cell specificity and reactivity toward a selected target in a non-WIC-restricted manner, taking advantage of the antigen-binding properties of monoclonal antibodies. The non-WIC-restricted antigen recognition confers T-cells expressing CARs on the ability to recognize an antigen independent of antigen processing, thus bypassing a major mechanism of tumor escape. In addition, when expressed in T-cells, CARs advantageously do not dimerize with endogenous T-cell receptor (TCR) alpha and beta chains.

In certain embodiments, the CAR sequence comprises an antigen binding domain, such as V_{H} and V_{L} gene segments of the antibody prepared according to the methods provided herein, and a T-cell signaling domain, which comprises, e.g. a hinge-CH2-CH3, a transmembrane domain and one or more cytoplasmic signaling domains. In certain embodiments, a transmembrane domain includes, but not limited to, transmembrane domains from CD8 alpha, CD28, CD3 epsilon, CD45, CD4, CD5, CD8, CD9, CD16, CD22, CD33, CD37, CD45, CD64, CD80, CD86, CD134, CD137, and CD154. In certain embodiments, the cytoplasmic signaling domains includes but not limited to intracellular co-stimulatory signaling domains from CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, CD54 (ICAM), CD152 (CTLA4), CD273 (PD-L2), CD274 (PD-L1), and CD278 (ICOS) and a primary signaling domain from TCR zeta, FcR gamma, FcR beta, CD3 gamma, CD3 delta, CD3 epsilon, CD5, CD22, CD79a, CD79b, and CD66d.

The present disclosure further provides uses of the CAR so produced in immunotherapy, such as in chimeric antigen receptor T-cell therapy (CAR-T).

Also provided herein is a method for producing a chimeric antigen receptor (CAR), comprising a step of expressing a first nucleic acid operably linked to a second nucleic acid, wherein the first nucleic acid encodes an antigen binding domain derived from the antibody or antigen-binding fragment thereof produced according to the method or the antibody described herein, and wherein the second nucleic acid encodes one or more T-cell signaling domains.

Immune cells such as T cells and Nature Killer (NK) cells can be genetically engineered to express CARs. T cells expressing a CAR are referred to as CAR-T cells. CAR can mediate antigen-specific cellular immune activity in the T cells, enabling the CAR-T cells to eliminate cells (e.g. tumor cells) expressing the targeted antigen. In one embodiment, binding of the CAR-T cells provided herein to cancer cells, resulting in proliferation and/or activation of said CAR-T cells, wherein said activated CAT-T cells can release cytotoxic factors, e.g. perforin, granzymes, and granulysin, and initiate cytolysis and/or apoptosis of the cancer cells.

In some embodiments, the T-cell activation domain of the CAR comprises a co-stimulatory signaling domain and a TCR signaling domain, which can be linked to each other in a random or in a specified order, optionally with a short peptide linker having a length of, for example, between 2 and 10 amino acids (e.g. glycine-serine doublet linker).

In some embodiment, the CAR further comprises a transmembrane domain. When expressed in cells, the antigen binding domain is extracellular, and the T-cell activation domain is intracellular.

In certain embodiments, the CAR comprises an antigen binding domain, a transmembrane domain, a costimulatory signaling region, and a TCR signaling domain, wherein the antigen binding domain specifically binds to an antigen and comprises an antigen-binding fragment of the antibodies provided herein.

The following examples are provided to better illustrate the claimed invention and are not to be interpreted as limiting the scope of the invention. All specific compositions, materials, and methods described below, in whole or in part, fall within the scope of the present invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1

### Materials and Methods

Materials:
LSM Lymphocyte Separation Medium (MP, cat.V0111A)
LLME: L-leucyl-L-leucine methyl ester (BacheM, cat.G-2550.0001)
Ham's F-12 Nutrient Mixture (Gibco, cat.11765047)
Heparin anticoagulation tube (BD, cat.367878)
Disposable blood collecting needle (BD, cat.367237)
IL2, Interleukin-2, lymphokine, TCGF (sinobiological, cat.11848-HNAY1-50) BCGF-1, BCGF1, BSF-1, BSF1, IL-4, Interleukin-4 (sinobiological, cat.GMP-11846-HNAE-100)
CD154, CD40 Ligand (sinobiological, cat.10239-H01H-50)
OX40L (sinobiological, cat.13127-H04H-100) Human ICOS Ligand/B7-H2/ICOSLG (Histag) (sinobiological, cat.11559-H08H-100)
Human ICOS/MUM/CD278 Protein (His & Fc Tag) (sinobiological, cat. 10344-H03H-100)
Human Interleukin-21/IL21 (sinobiological, cat.GMP-10584-HNAE-20)
Human BLyS/TNFSF13B/BAFF (sinobiological, cat.10056-HNCH-5)
Ephrin-B 1 (sinobiological, cat.10894-H08H)
Goat anti-Human IgG-Fc (HRP) (sinobiological, cat.SSA001-1)
Goat anti-Human IgM mu chain (HRP) (Abcam, cat.ab97205)
GlutaMAX™ Supplement (Gibco, cat.35050-061)
MEM NEAA (Gibco, cat.11140-050)
Sodium pyruvate (Gibco, cat.11360-070)
DMEM (no Glutamine, no Sodium Pyruvate, no HEPES) (Gibco, cat.11960-051)
Penicillin-Streptomycin, Liquid (Gibco, cat.15140122)
Recombinant Human Insulin (yuanye, cat. S31559-100 mg)
RPMI 1640 Medium (Gibco, cat.21875091)
DAPI (4′,6-diamidino-2-phenylindole; stock: 5 mg/ml in dH₂O; Thermo Fisher, cat. no. D1306)

TMB substrate (TIANGEN, cat.PA107-01)

FBS (GIBCO, cat. 10099141)
PBS (8117158)
E6446 dihydrochloride (MCE, cat. HY-12756A)
Anti-Human CD3 PE-Cy7 (eBioscience, cat. BG-05121-77-100)
Anti-Human CD21 PE (eBioscience, cat. 85-12-0219-42)
Mouse Anti-Human CD35-FITC (eBioscience, cat. 05-9600-02)
Anti-Human CD19 PerCP-Cy5.5 (eBioscience, cat. BG-11211-70-100)
Imaging reader (Biotek, cat.Cytation 5)
96 well Elisa plate (Corning, cat.9018)
Human IL1β (Novoprotein, cat. CG93)
Human CCL8 (Novoprotein, cat. CC95)
Human CXCL5 (Novoprotein, cat. CF14)
Mouse CXCL16 (Novoprotein, cat.CC44)
Recombinant Human CXCL13 (SB, cat.10621-HNAE)
Recombinant Human CCL4 (SB, cat.10899-H08Y)
Recombinant Human IL27 (SB, cat.10076-H085)
Human CXCL2 (Novoprotein, cat.0096)
Chicken OVA (sigma, cat.A5378-1G)
Total RNA isolation kit (Takara, cat.9767)

Methods:

Mice Immunization

Ovalbumin (OVA) was used as an antigen. Monoclonal antibodies against OVA were developed by sequentially immunizing BLAB/C mice. BALB/C mice were immunized via footpad route for all injections. The first injection was with OVA and admixed with Adju-Phos and CpG per mouse, followed by a final boost of 10 μg OVA in DPBS, without adjuvant. The BLAB/C mice were immunized on days 0, 3, 6, 10, 13, 17, 20, and 24 for this protocol and fusions were performed on day 29.

Tissue Isolation

The mice were anesthetized with isoflurane, the abdomen of those mice was opened under aseptic condition. The subcutaneous fat layer of the abdomen was harvested and the lymph nodes—inside were discarded, and the rest of those tissues were stored at -20° C.

Total RNA Isolation of Lymphatic Peripheral Adipose Tissue

Mice were immunized with chicken ovalbumin (OVA), and lymph nodes were harvested 30 days later. The adipose tissues associated with lymph nodes were dissected out. The total RNA is isolated by the total RNA isolation kit (Takara, cat.9767).

RNA-Seq

We prepared samples and send them to ANOROAD genome for RNA seq and analyze data through R language.

Preparation of Human Peripheral Blood Mononuclear Cells (PBMC)

Culture medium was prepared for PBMCs: (RPMI1640: DMEM: Ham's F12=1:1:2) (eRDF) supplemented with 10% FBS. Fresh PBMC was harvested from several healthy donors (about 40m1/time/person). PBMC was separated by density-gradient centrifugation as previously described in human monoclonal antibody book. Cell number was counted with a hemocytometer.

In Vitro Immunization (IVI)

PBMCs were washed and diluted with 10%FBS+eRDF. Cell density was adjusted to 1*10\hat{ }7cells/ml, treated with 0.25mM LLME for about 20min. Discarded supernatant, and re-suspended cell with 10%FBS+eRDF. Adjusted cell density to 9*10\hat{ }5cells/ml. Transfered cells suspension into 96 well plate, and added 2 ug/ml antigen, 10 ng/ml IL2, 50 ng/ml IL21, CD40L, ICOS, synthesized TLR7/8 agonist, respectively. Cultured the tissue for 7 days at 37° C., 5%CO₂. Changed half of the medium and added cytokine/activator cocktail on day7. Cultured the cells for 7-21 days at 37° C., 5%CO₂. Collected the supernatant on day 7, day 14, or day 21 for analysis of antibody production by ELISA, whereas the pellets are used for testing gene expression by PCR or RT-PCR. The collected cells were also tested in ELISpot assay for FACS analysis.

Measurement of the Antibody Level After Incubation with Antibody-Enhancing Composition

After day7 or dayl4 with addition of cytokines or antibody-enhancing composition and antigens, supernatants were harvested and added to antigen OVA-coated plates. After 2 hr incubation, a HRP-conjugated anti-human IgG or anti-human IgM was added, the amount of antigen-specific antibody was measured using TMB as substrate. The data represents the mean of 2 replicates; error bars represent SD. One representative data of 3 separate experiments is shown.

Flow Cytometry

We analyzed stained cells on an airell (BD) and processed flow cytometry data with FlowJo software (Tree Star). PBMCs were collected into Snap-lock microtubes. For analysis of T cells or B cells, tubes were kept at 4° C. unless mentioned otherwise. After centrifugation, cells were washed and resuspended in PBS. For analysis of T follicular helper cells, PBMCs were stained with antibodies of CD3-FITC (BD), CD4-PerCP-Cy™5.5 (BD), CXCR5-PE/Cy7 (Biolegend), and CD45RA-PE(eBioscience), respectively. T follicular helper cells were identified with CD3⁺CD4⁺CXCR5⁺CD45RA⁻. For analysis of GC like B-cells, PBMC was stained with antibodies of CD19-PE (eBioscience), GL7-Alexa Fluor 488 (eBioscience), Fas-APC (eBioscience). GC like B-cells were defined as CD19^{±}GL7⁺Fas⁺.

Reverse Transcription PCR

Quantitative RT-PCR was carried out with a BioRad iCycler and the 2−(ΔΔCT) method was used to calculate relative mRNA expression levels normalized to GAPDH.

Enzyme-Linked Immunosorbent Assay

Plates were coated with antigen at 5 μg/ml overnight at 4° C. and washed them in PBST (containing 0.5%Tween-20). The plates were blocked with 5% BSA before addition of cell culture supernatants and horseradish peroxidase (HRP)-conjugated detection antibodies (dilutions: 1 in 2,500 for HRP-conjugated IgG-specific antibody (Jackson) and HRP-conjugated IgM-specific antibody. TMB substrates solution was used for measurement.

Enzyme Linked Immunospot Assay (ELISpot)

Nitrocellulose-backed 96-well MAHAS4510 plates (Millipore) were coated overnight at 4° C. with (5 μg/mL) in 50 mM sodium bicarbonate buffer (pH 9.6). Plates were washed and blocked for 2 h at 37° C. with 10% fetal calf serum in RPMI1640. PBMCs were seeded at 3*10\hat{ }5 cells/well and incubated for 24 h at 37° C. Spot-forming cells (SFCs) were then detected using 2,000-fold diluted goat anti-human IgG antibody conjugated with horse radish peroxidase and incubate for 2 hr at 37° C. Ab binding was evaluated by the addition of TrueBlue substrate solution substrate (KPL, Gaithersburg, Md.).

Statistical tests with appropriate underlying assumptions on data distribution and variance characteristics were used. Except when noted other- wise, Two-way ANOVA were used to compare endpoint means of different groups. Regression and graphing were performed with Prism6 (GraphPad).

### Example 2

### Effects of the Antibody Production Stimulating Factors

As described in WO2018/205917, the in vitro expansion of PBMCs includes antibody-producing B cell, T cell, dendritic cells and adipocytes can form the germinal-center like structure in in vitro culturing system. IL2, IL21, ICOSL, ICOS, CD40L and toll-like receptor (TLR) agonists were proved to have stimulation effects on the antibody production in B cells.

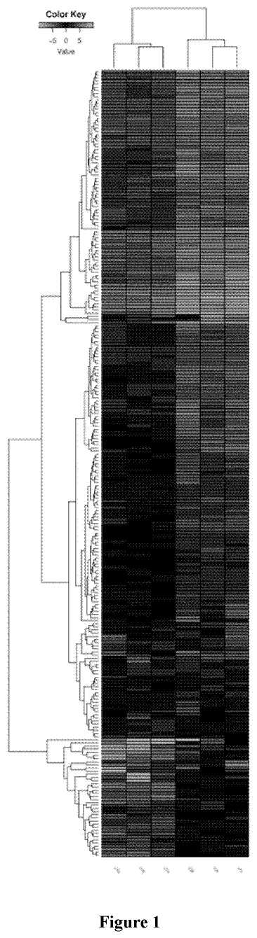

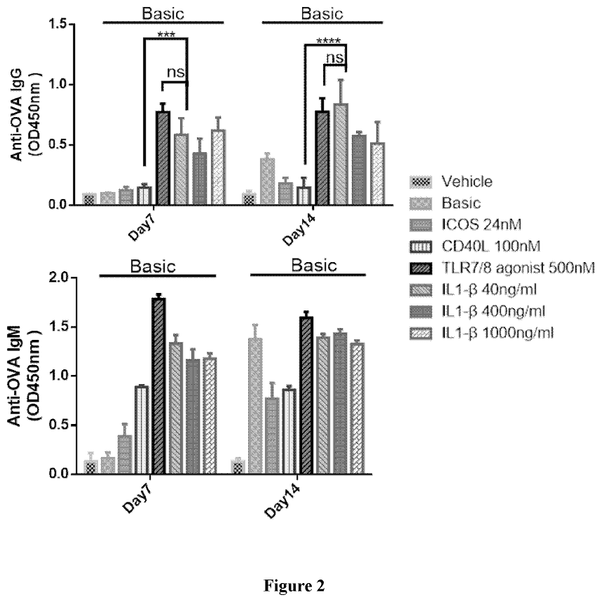

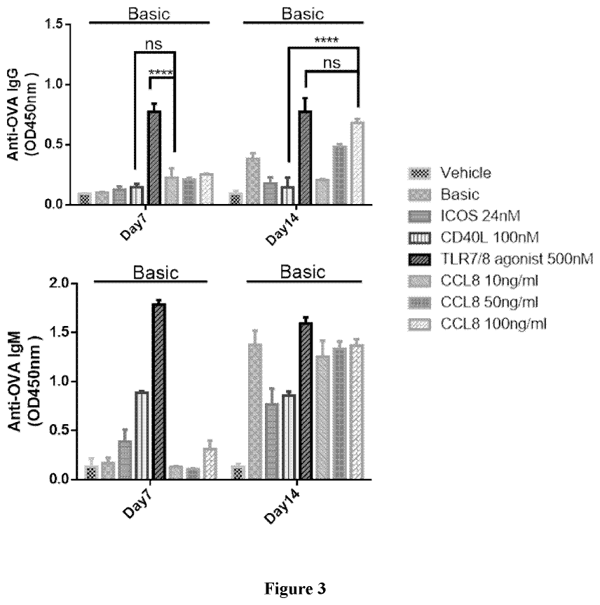

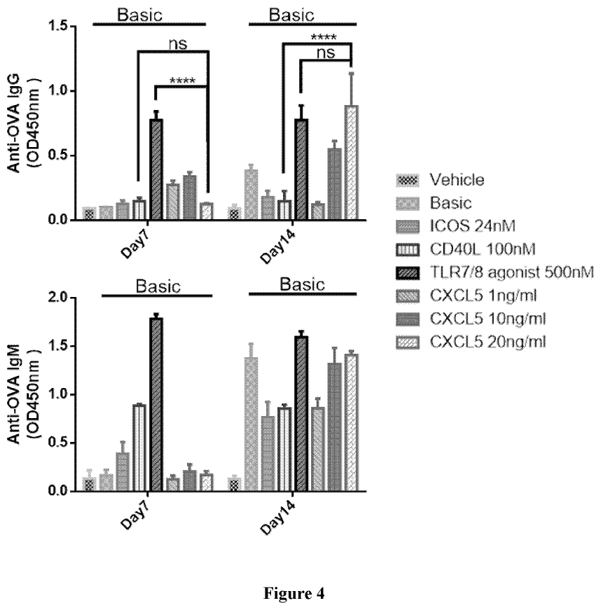

Specifically, in the in vitro antibody production system, IL2 and/or IL21 promotes the proliferation of the PBMCs, including B cell, T cell and dendritic cell populations (see FIGS. 1 and 3 of WO2018/205917); IL21 also promotes the class switch from IgM to IgG (see FIG. 3A-3B of WO2018/205917); both ICOSL and ICOS can induce antibody level produced by B cells, while ICOSL and CD40L synergistically enhance the IgG production, rather than ICOSL or CD40L alone (see FIG. 2A-2B and 4A-4B of WO2018/205917).

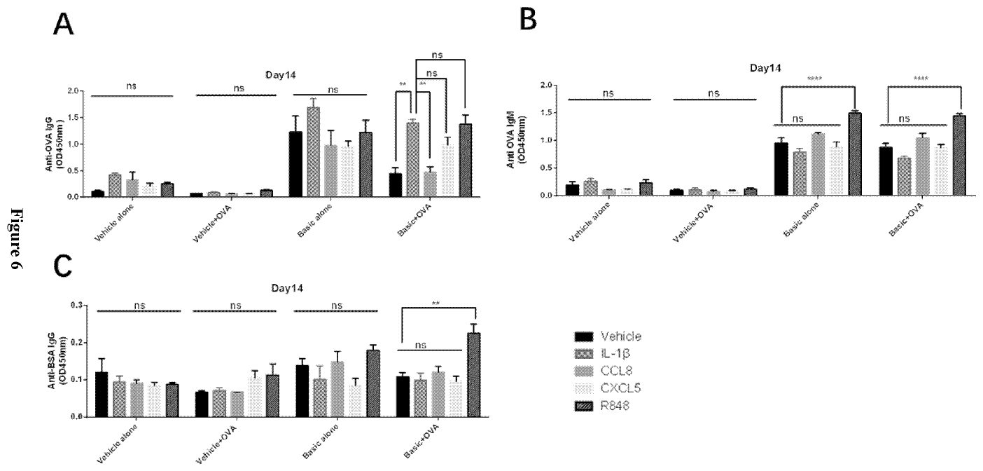

In addition, the TLR agonists (such as synthesized TLR7/8 agonist and TLR9 agonist) also act as a key factor for the enhanced antibody production, as indicated by the result that TLR7/8 agonist was far more effective than CD40L in stimulating the production of anti-OVA antibody (see FIG. 6A of WO2018/205917). For IgG antibodies, the TLR7/8 agonist was more effective at 14 days in vitro (about 3.5 and 10.0 fold higher than CD40L with 50 nM and 500 nM of the TLR agonist, respectively) than at 7 days and 21 days in vitro (FIG. 6A)

FIG. 10 of WO2018/205917 shows the TLR9 agonist CpG ODN (2 μg/ml) elicited similar effects as CD40L in stimulating the production of anti-OVA antibodies at day 14 after addition of the factors (for both IgG and IgM).

The expression of AID (involved in B cell affinity maturation by inducing hyper-mutation in antibody genes) and BLIMP-1 (indicates proliferation and differentiation of active B cell) were increased by TLR7 (imiquimod, 500 nM) agonist or TLR7/8 agonist. Both FIG. 8 and FIG. 9 of WO2018/205917 show that TLR7 agonist and synthesized TLR7/8 agonist are far superior to CD40L in stimulating the expression of AICDA and BLIMP-1. Also indicated in FIG. 8 and FIG. 9 of WO2018/205917 is the superior ability of TLR7 in inducing enriched antibody variants via hypermutation, and higher affinity of the antibody, as compared with CD40L.

FIGS. 11A-11C of WO2018/205917 show that low concentration of TLR9 antagonist E6446 (e.g. 0.02 μM and 0.2 μM) promotes the effects of TLR7/8 agonist on antibody IgG production, indicating synergistic effects between TLR7/8 agonist and low concentration of TLR9 antagonist E6446. However, high concentration of E6446 (10 μM) reverses the effects of TLR7/8 agonist in both 7-day and 14-day cultures, for both IgG and IgM production (see FIGS. 11D-11F of WO2018/205917).

FIG. 11G of WO2018/205917 shows stimulation of PBMCs with TLR7/8 agonist after blocking TLR9 (0.02 μM and 0.2 μM E6446 dihydrochloride, respectively) in vitro resulted in a significant increase in IgG responses and cell activity, indicating that TLR7/8 agonist facilitates the generation of dendritic cells, which is partially reversed by high concentration of TLR9 antagonist E6446.

FIG. 12A-12I of WO2018/205917 shows that IL2 and IL21 co-stimulated with ICOS, CD40L, or synthesized TLR7/8 agonist respectively resulted in synergistic, complimentary effects on enhanced IgG production.

FIGS. 13A-13F of WO2018/205917 show that ICOS (24 nM, 55 nM, 100 nM), CD40L (10 nM, 24 nM, 55 nM), TLR7/8 agonist (0.1 nM, 50 nM, 500 nM) regulated IgG and IgM responses in a dose-dependent manner in the presence of 4 μg/ml OVA, 10 ng/ml IL2, and 50 ng/m1 IL21.

### Example 3

### Identification of Adipose Tissue-Derived Proteins that Enhance Antibody Production

In searching for adipose tissue-derived proteins that could stimulate antibody generation, RNA seq analyses were performed to identify genes up-regulated in lymph node-associated adipose tissues after immunization with an antigen. Total RNA from the adipose tissues with or without immunization were subject to differential gene expression analysis through RNA-seq techniques.

Using “2-fold increase” as a cut-off criterion, a total of 273 genes were up-regulated from the adipose tissues after immunization (see FIG. 1). Among those, 69 genes encode secretory proteins. A detailed analysis indicates that these secretory proteins fall into three categories: cytokines, chemokines, and cell-adhesion molecules, which are listed in Table 1.

TABLE 1
A complete list of adipose tissue-derived secretory proteins
significantly up-regulated after immunization.

| | | | Log 2 Fold | | |
|---|---|---|---|---|---|
| Classification | Gene Name | Fold Change | Change | pval | padj |
| Chemokine | Cxcl5 | 37.76697 | 5.239053 | 3.96E−07 | 0.000151 |
| | Ccl4 | 16.13672 | 4.012275 | 5.92E−05 | 0.009147 |
| | Cxcl2 | 15.78797 | 3.980753 | 0.000388 | 0.037344 |
| | Ccl6 | 9.398073 | 3.232365 | 9.44E−10 | 1.00E−06 |
| | Ccl9 | 7.793868 | 2.96234 | 5.34E−07 | 0.000191 |
| | Cxcl9 | 7.028796 | 2.813278 | 1.20E−07 | 5.95E−05 |
| | Ccl11 | 3.651545 | 1.868507 | 1.46E−05 | 0.002844 |
| Interleukin | Il1f9 | 31.53936 | 4.979082 | 0.000184 | 0.021873 |
| | Il10 | 11.79523 | 3.560131 | 0.000488 | 0.043515 |
| | IL1β | 9.869144 | 3.302925 | 4.86E−06 | 0.001233 |
| | Il27 | 9.048462 | 3.177673 | 0.00059 | 0.049352 |
| | Il33 | 3.805356 | 1.928032 | 3.14E−06 | 0.000879 |
| | Il18bp | 3.47187 | 1.795713 | 5.33E−05 | 0.00828 |
| Cell- | Itgam | 17.83271 | 4.156454 | 4.27E−06 | 0.001133 |
| adhesion | Siglecf | 12.26847 | 3.616883 | 3.37E−05 | 0.005675 |
| molecule | Adam8 | 11.18791 | 3.483869 | 4.20E−06 | 0.001122 |
| | Chl1 | 7.698579 | 2.944592 | 0.000173 | 0.021025 |
| | Sirpa | 4.740933 | 2.245171 | 6.97E−06 | 0.001578 |
| | Nrcam | 4.740909 | 2.245164 | 0.000229 | 0.025499 |
| | Emilin2 | 4.223717 | 2.078513 | 1.07E−06 | 0.000351 |
| | Emilin1 | 3.249264 | 1.700113 | 0.000183 | 0.021848 |
| | Tubb6 | 3.073325 | 1.6198 | 9.11E−05 | 0.012933 |
| | Parvb | 2.952176 | 1.561779 | 0.000479 | 0.04294 |

### Example 4

### Effects of Adipose Tissue-Derived Secretory Proteins (ADSPs) in Antibody Production

Previous work has established that specific antibody could be generated in vitro by applying an antigen (e.g. OVA) to human peripheral blood mononuclear cells (PBMC), and incubate with a combination of regulatory factors for one or two weeks. Two required factors are IL2 and IL21 (together named “basic” or “basic medium” here). ICOS, CD40L, TLR7/8 agonists could further stimulate antibody production. To test the ADSPs that were up-regulated in Table 1, further experiments were performed.

In all the following performed experiments, statistical analysis was performed using ANOVA followed by ad hoc tests. The data represents the mean of 3 replicates, error bars represent SD. ***, p<0.001, ****, p<0.0001.

1.1. Effects of Interleukins

An exemplary cytokine, IL1β, was tested. PBMCs were incubated in vitro with OVA, basic, and either some of the known stimulating factors ICOS, CD40L, TLR7/8 agonists or IL1β (in different doses) (1.5*10\hat{ }5 cell/well, 96 well plate). After 7 or 14 days, supernatants were harvested, and IgG/IgM antibody production was measured by ELISA assay. A representative data of 3 separate experiments is shown in FIG. 2. In general, the effect of IL1β is similar to TLR7/8 agonist but better than ICOS and CD40L in stimulating antibody production.

1.2. Effects of Chemokines

1.2.1 CCL8

The experiments were performed exactly the same as described above in Example 3, section 1.1, except CCL8 was used instead of IL113 (see FIG. 3). For IgG production, the effect of CCL8 is inferior to TLR7/8 agonist but better than ICOS and CD40L.

1.2.2 CXCL5

The experiments were performed exactly the same as described above in Example 3, section 1.1, except CXCL5 was used instead of IL1β (see FIG. 4). For IgG production in vitro for 14 days, the effect of CXCL5 is dose-dependent, and at 20 ng/ml, CXCL5 is inferior to TLR7/8 agonist but better than ICOS and CD40L.

1.3 Effects of Other ADSPs

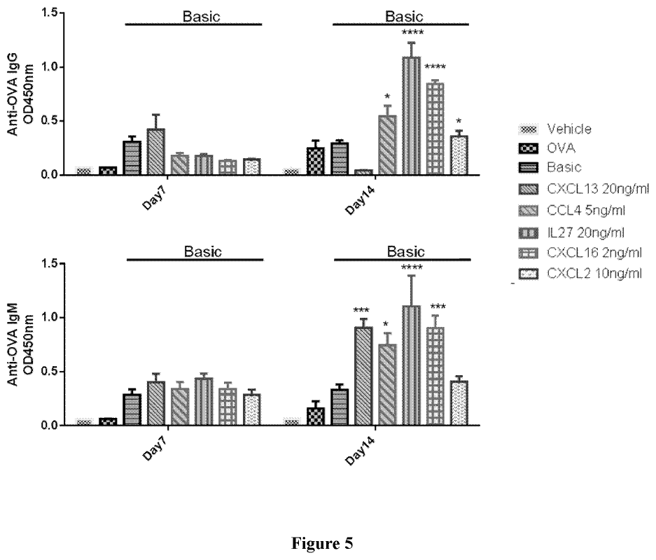

The experiments were performed exactly the same as described above in Example 3, section 1.1, except CXCL13, CCL4, IL27, CXCL16, CXCL2 were used instead of IL 1β, respectively (see FIG. 5). For IgG production in vitro for 14 days, the effects of CCL4, IL27, CXCL16, CXCL2 are better than Basic alone. For IgM production in vitro for 14 days, the effects of CXCL13, CCL4, IL27, CXCL16 are better than Basic alone.

### Example 5

### Antigen Specific Antibodies Generated in the Presence of ADSPs in In Vitro Immunization

To investigate individual function of the components in the system of in vitro immunization, the experiments were done exactly the same way as that in Example 3, section 1.1. Cultured PBMC were treated with IL-1β, CCL8, CXCL5 or the TLR7/8 agonist R848 (1.5*10\hat{ }5 cell/well, 96 well plate) for 14 days. The culture medium was harvested, the production of OVA-specific IgG (FIG. 6A) or IgM (FIG. 6B) antibody was measured by ELISA assay. Non-specific antibodies were measured by BSA-coating ELISA plate to determine whether and how much they bind BSA (FIG. 6C). A representative data of 3 separate experiments is shown. Statistics: ANOVA followed by post hoc analyses. The data represent the mean of 3 replicates, error bars represent SEM. **, p<0.01, ****, p<0.0001.

Based on the antibodies produced (anti-OVA IgG, anti-OVA IgM, and anti-BSA IgG) in the presence or absence of the antigen OVA, the antibodies generated under “basic medium” condition are those produced without antigen stimulation, indicating that they are non-specific and the “basic medium” may activate the antibody-producing B cell non-specifically (see the third panels “basic alone” in FIGS. 6A-6C), while IL-1β promotes generation of anti-OVA IgG in a high level compared with that of anti-OVA IgM and anti-BSA IgG in the presence of the antigen OVA, indicating that IL-10 specifically stimulates the activation of OVA-specific B cell, i.e. antigen-specific B cell (see the fourth panels “basic +OVA” in FIGS. 6A-6C). CCL8 and CXCL5 can similarly stimulate the activation of OVA-specific B cells in generating anti-OVA IgG and anti-OVA IgM antibodies.

### Example 6

### Expression of ADSPs in Mice

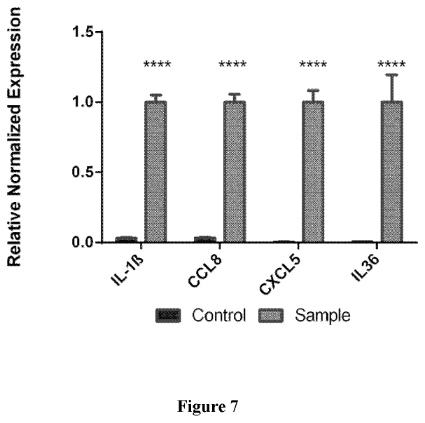

The experiment is performed to investigate the expression level of IL-1I3, CCL8, CXCL5, 11,36 in adipose tissues derived from lymph nodes, with or without immunization with OVA (10 ₁1g/time, 8 times, see FIG. 7). The levels of mRNA for different factors were determined by quantitative RT-PCR and quantitative analysis by the 2-(ΔΔCT) method. Adipose tissues were collected from healthy BALB/C mice with (sample) or without (control) OVA immunization. ACTB expression was used for the normalization. A representative data of 3 separate experiments are shown. In FIG. 7, statistical analysis was performed using ANOVA tests. ****, p<0.0001.

While the disclosure has been particularly shown and described with reference to specific embodiments (some of which are preferred embodiments), it should be understood by those having skill in the art that various changes in form and detail may be made therein without departing from the spirit and scope of the present disclosure as disclosed herein.

What is claimed is:

1. A method for producing an antibody or an antigen-binding fragment thereof specifically binding to an antigen of interest, the method comprising
mixing the antigen, an antibody-generating cell composition (AGC), and an antibody-enhancing composition in a medium to form a mixture,
cultivating the mixture,
obtaining the antibody from the mixture,
wherein the AGC comprises at least one B cell and at least one additional type of cell derived from peripheral blood mononuclear cells (PBMCs), and the antibody-enhancing composition comprises one or more adipose tissue-derived secretory proteins (ADSPs).

2. The method of claim 1, wherein the antibody-enhancing composition further comprises IL2 and/or IL21.

3. The method of claim 1, wherein the adipose tissue-derived secretory protein comprises one or more cytokines and/or one or more cell-adhesion molecules.

4. The method of claim 3, wherein the cytokines comprise one or more interleukins and/or one or more chemokines.

5. The method of claim 4, wherein the interleukins are selected from a group consisting of IL-1β, IL1f9, IL10, IL27, IL33 and IL18BP.

6. The method of claim 4, wherein the chemokines comprise one or more CC-chemokines selected from a group consisting of CCL4, CCL8, CCL6, CCL9 and CCL11, or one or more CXC-chemokines selected from a group consisting of CXCL2, CXCL5, CXCL16, CXCL9 and CXCL13.

7. The method of claim 3 or claim 4, wherein the cytokines are selected from a group consisting of IL-1β, CCL8 and CXCL5.

8. The method of claim 3, wherein the cell-adhesion molecules are selected from a group consisting of ICAM1, CSF3r, Itgam, Siglecf, Adam8, Chl1, Sirpa, Nrcam, Emilin2, Emilinl, Tubb6, and/or Parvb.

9. The method of any one of claims 1-8, wherein the ADSP is derived from an adipose tissue.

10. The method of any one of claims 1-9, wherein the AGC comprises at least one B cell and at least one T follicular helper cell.

11. The method of any one of claims 1-9, wherein the AGC comprises at least one B cell and at least one dendritic cell.

12. The method of any one of claims 1-9, wherein the AGC comprises at least one B cell, at least one T follicular helper cell and at least one dendritic cell.

13. The method of any one of claims 1-12, wherein the AGC further comprises at least one adipocyte.

14. The method of any one of claims 1-13, wherein the AGC comprises PBMCs.

15. The method of claim 14, wherein the PBMCs are isolated from a blood sample, derived from human hematopoietic stem cells (HSCs), derived from induced pluripotent stem cells (iPSCs) or derived from umbilical cord blood.

16. The method of any one of the preceding claims, wherein the antibody-enhancing composition further comprises a co-stimulator, a toll-like receptor (TLR) agonist, a CpG oligodeoxynucleotide (CpG ODN), an anti-apoptotic protein, a TNF, an interferon (INF), a lipid, avasimid, EFNB1, EPHB4, Plexin B2, Semaphorin 4C, BLIMP-1, IRF4 or any combination thereof

17. The method of claim 16, wherein the co-stimulator comprises CD40, CD40L, ICOSL, ICOS, APRIL, B cell activating factor of the TNF family (BAFF), OX40, and/or OX40L.

18. The method of claim 16, wherein the CpG ODN comprises CpG2006, and/or D/K CpG.

19. The method of claim 16, wherein the anti-apoptotic protein comprises Bcl-2, Bcl-6, Bcl-XL, Bcl-w, Mcl-1, and/or an analog thereof.

20. The method of claim 16, wherein the TLR agonist comprises a TLR1 agonist, a TLR2 agonist, a TLR3 agonist, a TLR4 agonist, a TLRS agonist, a TLR6 agonist, a TLR7 agonist, a TLR8 agonist, a TLR7/8 agonist and/or a TLR9 agonist.

21. The method of any one of the preceding claims, wherein the adipose tissue-derived secretory protein enhances antibody production by the AGC, activation and differentiation of the B cell in the AGC, and/or maturation of the B cell in the AGC.

22. The method of any one of the preceding claims, further comprising isolating the antibody generated in the mixture.

23. The method of claim 23, further comprising obtaining a nucleic acid sequence encoding a variable region of the antibody.

24. The method of claim 24, further comprising introducing the nucleic acid sequence into a host cell under a condition suitable for expressing the antibody or the antigen-binding fragment thereof.

25. The method of any one of the preceding claims, further comprising evaluating if the antibody specifically binds to the antigen of interest.

26. The method of any one of the preceding claims, wherein the ADSP is present at a concentration of at least 1 ng/ml, 10 ng/ml, or 50 ng/ml.

27. The method of claim 2, wherein IL2 is present at a concentration of at least 10 ng/ml.

28. The method of claim 2, wherein IL21 is present at a concentration of at least 50 ng/ml.

29. The method of claim 24, wherein the ADSP is present for at least 1 day.

30. The method of claim 25, wherein the IL2 is present for at least 1 day.

31. The method of claim 26, wherein the IL21 is present for at least 1 day.

32. A method for inducing proliferation of antibody-generating cell composition (AGC), B cell activation and differentiation, B cell maturation, and/or promoting class switch in an AGC to produce IgG, wherein the method comprising cultivating the AGC in a medium comprising IL2, an adipose tissue-derived secretory protein (AD SP), and/or IL21.

33. The method of any one of the preceding claims, wherein the antibody is a fully human monoclonal antibody.

34. A method for producing an antibody or antigen-binding fragment thereof specifically binding to an antigen of interest, the method comprising:
mixing the antigen, an antibody-generating cell composition (AGC), and an antibody-enhancing composition in a medium to form a mixture,
cultivating the mixture,
obtaining the antibody from the mixture,
wherein the AGC comprises at least one B cell and at least one additional type of cell derived from peripheral blood mononuclear cells (PBMCs), and the antibody-enhancing composition comprises IL2, IL21, and one or more adipose tissue-derived secretory proteins (ADSPs).

35. The method of claim 34, further comprising
obtaining a nucleic acid molecule encoding a variable region of the antibody from the mixture; and optionally
introducing the nucleic acid molecule into a host cell under a condition suitable for expressing the antibody or the antigen-binding fragment thereof.

36. The method of claim 35, further comprising
isolating the antibody or the antigen-binding fragment thereof secreted by the host cell.

37. A composition comprising isolated antibody-generating cell composition (AGC) comprising at least one B cell and at least one additional type of cell derived from peripheral blood mononuclear cells (PBMCs), an antibody-enhancing composition, and a medium.

38. The composition of claim 37, further comprising an antigen of interest.

39. The composition of claim 37, wherein the antibody-enhancing composition further comprises IL2 and/or IL21.

40. The composition of claim 37, wherein the AGC comprises at least one B cell and at least one T follicular helper cell.

41. The composition of claim 37, wherein the AGC comprises at least one B cells and at least one dendritic cell.

42. The composition of claim 37, wherein the AGC comprises at least one B cell, at least one T follicular helper cell and at least one dendritic cell.

43. The composition of claim 37, wherein the AGC comprises PBMCs.

44. The composition of any one of claims 40-43, wherein the AGC further comprises at least one adipocyte.

45. The composition of claim 37, wherein the antibody-enhancing composition comprises one or more antibody-enhancing factor selected from the group consisting of ADSP, CD40L, ICOSL, ICOS, TLR agonist and any combination thereof

46. A method for identifying an antibody-enhancing factor for in vitro immunization, comprising:
a) isolating total RNA from a cell derived from a lymph node of an animal immunized with an antigen of interest;
b) comparing the RNA levels of the total RNA isolated from the step a) with that of a control animal without immunization to determine a gene which encodes a protein and whose expression level is upregulated;
c) cultivating PBMCs in a medium comprising the antigen of interest, IL2, IL21 and the protein;
d) identifying the protein as an antibody-enhancing factor for in vitro immunization if the protein enhances antibody production.

47. The method of claim 46, wherein the cell is an adipocyte, a T follicular helper cell, a B cell or a dendritic cell.

48. The method of claim 46, wherein the protein is expressed by the adipocyte, the T follicular helper cell, the B cell or the dendritic cell.

49. A method for producing a chimeric antigen receptor (CAR), comprising a step of expressing a first nucleic acid operably linked to a second nucleic acid, wherein the first nucleic acid encodes an antigen binding domain derived from the antibody or antigen-binding fragment thereof produced according to the method of claim 1-36, and wherein the second nucleic acid encodes a T-cell signaling domain.

50. A method of treating a cancer in a subject comprising:
expressing in a T cell a first nucleic acid operably linked to a second nucleic acid, wherein the first nucleic acid encodes an antigen binding domain derived from the antibody or antigen-binding fragment thereof produced according to the method of any one of the claims 1-36, and wherein the second nucleic acid encodes a T-cell signaling domain; and
administering the T cell to the subject.

51. The method of claim 48, wherein the T cell is obtained from the subject.
